SEQUENCE LISTING (9 sequences)

SEQ ID NO: 1 - DNA, 828 bp, Glycine max
  source 1..828: mol_type = other DNA
atgaccctccccaccaatctcaacttcttctatgtgttcctcccaatgctcattctctattcctcttccttccttgcatcagcttctgattctaaagctgaagatgaaaaatttacctacgctataggaagtagcacaggaccagagaattggtggcatgtcaaccaaaaatggaaaacatgtggggatggaaaactacaatctcccattgatctgcttgaccaaagggttcaagagcttcctcaattgggtaaacttaagaaagcttacaaatcagctcctgctgtcctaaagaatagtggtcatgacatcgtgctggaatggaaaggagatgcaggccatcttaacataaatgaaacttactacaatctgattcaatgtcattggcatacaccttcagagcacacgttgaatggaacaaagtttgacttggaactgcatgcagtccataaatcctctaagggagagtttgctgttattggaatattgtataaaattggttctccagatccctttttttcaaagctgctcaatgacataaaatcaagtgtggacaaggatatagatgtaggagtaatcaacccaagagagatcaagtttaaaagcagaccgtactacagatatgttggttctcttacaactccaccatgcactgaaggtgttgtttggacaattgtgaagaaggtcaggacagtctcaagcgagcaattaagtgccttgaaaggagctgttcatcatggatatgaggaaaatgcaaggccaacgcaggaacttggtggaagacaagtttggttatatggacctatggagaatgaaaagcctacttaa

SEQ ID NO: 2 - DNA, 1932 bp, Thalassiosira weissflogii
  source 1..1932: mol_type = other DNA
atgaaccaatccaacacatcctcatctacctcttctgcctcgctaactcccgatcaaatcgtcgcggccctccaagagcgaggatggcaagccgaaatcgtcacggaattctccctcctcaacgaaatggtcgacgtcgatcctcaaggcatcttgaaatgcgtggacggacgaggatccgacaacacccaattctgcgggccgaaaatgcccggaggaatttatgccattgctcacaatcgaggagtcaccacgttggaggggttgaagcaaatcaccaaggaagtggccagcaagggccacgttccctccgttcacggagatcactcttccgacatgttgggatgcggattcttcaaattgtgggtcacgggtcgtttcgacgacatgggatacccccgtccgcaattcgacgccgatcagggagccaaggccgtcgagaatgccggaggcgtcatcgagatgcatcacggatctcatgccgaaaaggtggtatacatcaacttggtggagaacaaaacgttggagccagacgaagacgatcaacgcttcatcgtggatggatgggccgcgggaaagttcgggttggacgtgcccaaattcttgatcgcggcggcggccacggtggagatgctcggagggcccaaaaaagccaagatcgtcattccgtccatttctcccgcccagatcgccgaggccctacaaggccggggatgggacgccgagatcgttaccgatgcgagtatggcgggccagttggtggacgttcgaccggaaggcattctcaagtgcgtggacggacgggggtcggataatactagaatgggtgggcccaagatgcccggtgggatttatgccattgctcataatcgaggcgtgacttccatcgaggggttgaagcaaatcaccaaggaagttgctagcaagggccaccttccctccgttcacggagatcattcctcggatatgctcggatgtggattcttcaaactttgggtcaccggaagattcgacgacatgggatacccccgtccgcaattcgacgccgatcaaggcgccaacgccgtcaaagacgccggtggaatcatcgaaatgcatcacggatctcacaccgaaaaggtcgtctacatcaacctcctggccaacaagaccctcgaacccaacgaaaacgaccaacgcttcatcgtcgacggatgggccgccgacaaattcggattagacgtgcccaaattcctcatcgctgcggcggccacggtggaaatgctcggagggcccaaaaatgccaagatcgtcgttccttccatcactcctccgcaaatcgtttcggcgttgcgaggacgaggatggaaggcctccatcgtcaaggcctcgacgatgtcgtcggaattgaaacgcgtcgatccgcaggggattttgaagtgcgtcgacggcagagggtccgacaacacccaattcggcggaccgaaaatgcccggtggaatttacgccatcgctcacaatcgaggcgtcacgaccttggaaggtctcaaagacattacccgcgaagtcgcctcgaaaggccacgtgccctccgtgcacggggatcattcctccgatatgctcggatgcggattcttcaagttgtggctgaccggtcgattcgacgacatgggatatccccgaccggaattcgacgccgatcagggtgccctggccgtacgggccgccggaggagtcatcgagatgcatcacggaagtcacgaagagaaggtggtgtacatcaatctcgtgagcggaatgacgttggaacccaacgagcacgatcagaggttcatcgtcgatggatgggcggcgagcaagtttgggttggatgtggtcaagtttttggtggcagcggcggcgacggtggagatgctcggtggacccaaaaaagccaagattgttattccttaa

SEQ ID NO: 3 - DNA, 918 bp, unidentified
  source 1..918: mol_type = other DNA
atgggaagaattgctagaggctcaaaaatgagttctctcattgtgtctttgcttgtagtattggtgtcactcaatttggcttccgaaaccacagctaaatacacttactcctctccaccacctcccgagcattctcctccaccgccggagcattctcctcctccgccttaccactacgaatccccgcccccgcctaaacattctccaccaccacctacaccggtttacaagtacaagtctccaccgcctcctatgcattctcctccaccgccttatcattttgagtctccacctccaccaaaacattctccaccaccaccaacgccggtttacaagtacaaatctccaccaccacctaaacattctcctgcaccagtgcatcattataaatacaagtctccaccaccaccaacaccggtttataagtataaatctccaccaccaccaaagcattctcctgcaccagaacatcactataagtacaagtctccaccaccacctaagcattttcctgcaccagaacatcactataagtacaagtacaagtctccaccaccaccaacaccggtctacaagtataaatctccaccacctccaacaccggtctacaagtacaagtctccaccaccacccaagcattctcccgcaccagtacaccattacaagtacaagtctccaccaccaccaactccagtttataaatctccaccaccacccgaacattccccaccaccaccaacaccggtctacaaatacaagtctccaccaccaccaatgcactctccaccaccaccaacaccagtttacaagtacaagtctccgccaccaccaatgcactctcccccaccaccagtttactctccaccaccacccaaacatcactactcctatacgtcacctcctcctcctcaccactac

SEQ ID NO: 4 - DNA, 67 bp, Pisum sativum
  source 1..67: mol_type = other DNA
atgaagtgttggagattgtcggcgattctgtttctagggtttatgttgactagcttgtcaacggcgt

SEQ ID NO: 5 - DNA, 102 bp, Pisum sativum
  source 1..102: mol_type = other DNA
atgacttgggctgctttttggcttgctctcgtcgttttgattgccttagctatgattgcaggagggggattccttgtgtataaatatagaattaggcaataa

SEQ ID NO: 6 - DNA, 557 bp, Glycine max
  source 1..557: mol_type = other DNA
tgtattgctgctgaaatcacatggagtaattgtataaccatatcatctaatgccaaaacattttcttttattgcggtcttagtccaaagtgtggacttgtaggatatgctatacaacagttatacttttgaaaagacaattcagctactaataaataataattaatcctaaagctgaagaaattattactattattatgaaaataaataaatgtaattgctgcttggtggataggatgaaatgaaggacaagacctaagaaacatgtccgcacgtttttcacttacgtaatgatattcaattcctggatcaatatattcctacaaacattgtttcaaacaaaaaaataaaaggaaacaagagtaatactgtttgacttaagagattagagatgttatcttcgtggggatcacgaattgaagacaaatgagcgacgtgctattggaattggtccattggacgttatatataaggatacgtgtaccagtcaatgagcacgtaccctttagcattaatcctacatcacctagagaaaaccacacagccaaaaatttaaca

SEQ ID NO: 7 - DNA, 1993 bp, Glycine max
  source 1..1993: mol_type = other DNA
gtgatgttcttggccactggtaggtggtgcggttggctctcttaatgtatttttaaaaaaaaatagtttaggtccctttagttgttgtgtcggtcccatgggcgagcagtatatcctgaccataaacaaaagcattggaccaaggggaaaaaaacagcacagggacaaaatggcaaactacatcttaagctcaagacacaatacatgcttcagtttgattttttgagaaaaaattccttttaaataaaattaagtaaaattttgattataaattataaattttaacgttctaagattgagtaagcgataaaaatctaaattgaaaaggatctctacattggaaatgcccccaatatataagcgaaaaacaatacaacttctgtcttgtgtagcaaacgtttcccgcagagaattaatcattagaattttgctcttggataaattgtttcattcgttagtaaaataaagaaaacttatgttgtaaagtcgatttcgcattcacaggaacataaccgtgacaaatatgatgtctgtttattttaaataacaaaaattaagcacacatgtatattaaaaaaattattctacggatgatcatcattttcagataggtaatacgggtacaaatacatgtacggtttgtgaattgtacgtgtagataagcagttaatctatatcccattttatattattgttacgagattttcattaaatagacatggggtactcctttgtgacattgacatgacaaaaaactctgtatccatgtgtatttgttcgactctgtttcgattttgacgaataatacatgagttggtcgggtatggatatagttttttctgataaccaaaaatcagatacggatatgagattactacatccttcctgaccccgaacccgtcccgccacttaatataagtagttaaaaataaatgtttaacaatcaattcattttttctaaaatcaaatttttaatattttttttataaaaaaatatttttctaatttaaatcagatatagaacgggattggagatatccaatacccaacaggtacaaagataaataaagataaataataaattttaacctataagatacaagaacatgttcaaaaatcgtcatttccatgtctcattctaacatagttattgatatatgttgcataaatagaaagctagcgtaccaaaactctaaaaaaatgttcttaatttcttactcaactgaaaaattaacaaaccctaacaatatattaaagatggaatcgtcgatcatagaaaaactttgctgggaaaaaaaaacagacaagcatcattatatagaccatatgcatgctgtgtacggagtattgtttttgcaccacggggtgattgaaattgaaccgttagcgtcattgtaatatgcaaaacggagtcgtcagctagctcatttatgtctgcatcttcttcacatctcaccaccattaaaaaaatagtagtctcgcagaaatatcttttctgcgcaccgcagatcactgagtactgcctaacttacatgtgcatgaacgactacaatggcgccgagttaggcttataaatcaaattacacacagttcatagaaaatccaaccctattattattctgtctacatttaacccacttgagctcttttttcttttcttaatctgcaggtgaactctttttcttttctaagctatagaaagtagaaaccgcatataattaattaattataatttaggtgtcactctcttccttgttaaagaattaattaacctagctagagacatgtctataagagaggactaacggtgaaggtttaagcctctttggcctattctttatcgtgagagttttgcctttatttctcagccatgtttccttcttttccagcttaaaccgctaccctacaaaacctttcacaattctctttcttcctagctatctctttctttctgtctacattgacctagctagctacaaaccctgcattaacc

SEQ ID NO: 8 - protein, 275 aa, Glycine max
MTLPTNLNFFYVFLPMLILYSSSFLASASDSKAEDEKFTYAIGSSTGPENWWHVNQKWKTCGDGKLQSPIDLLDQRVQELPQLGKLKKAYKSAPAVLKNSGHDIVLEWKGDAGHLNINETYYNLIQCHWHTPSEHTLNGTKFDLELHAVHKSSKGEFAVIGILYKIGSPDPFFSKLLNDIKSSVDKDIDVGVINPREIKFKSRPYYRYVGSLTTPPCTEGVVWTIVKKVRTVSSEQLSALKGAVHHGYEENARPTQELGGRQVWLYGPMENEKPT

SEQ ID NO: 9 - protein, 643 aa, Thalassiosira weissflogii
MNQSNTSSSTSSASLTPDQIVAALQERGWQAEIVTEFSLLNEMVDVDPQGILKCVDGRGSDNTQFCGPKMPGGIYAIAHNRGVTTLEGLKQITKEVASKGHVPSVHGDHSSDMLGCGFFKLWVTGRFDDMGYPRPQFDADQGAKAVENAGGVIEMHHGSHAEKVVYINLVENKTLEPDEDDQRFIVDGWAAGKFGLDVPKFLIAAAATVEMLGGPKKAKIVIPSISPAQIAEALQGRGWDAEIVTDASMAGQLVDVRPEGILKCVDGRGSDNTRMGGPKMPGGIYAIAHNRGVTSIEGLKQITKEVASKGHLPSVHGDHSSDMLGCGFFKLWVTGRFDDMGYPRPQFDADQGANAVKDAGGIIEMHHGSHTEKVVYINLLANKTLEPNENDQRFIVDGWAADKFGLDVPKFLIAAAATVEMLGGPKNAKIVVPSITPPQIVSALRGRGWKASIVKASTMSSELKRVDPQGILKCVDGRGSDNTQFGGPKMPGGIYAIAHNRGVTTLEGLKDITREVASKGHVPSVHGDHSSDMLGCGFFKLWLTGRFDDMGYPRPEFDADQGALAVRAAGGVIEMHHGSHEEKVVYINLVSGMTLEPNEHDQRFIVDGWAASKFGLDVVKFLVAAAATVEMLGGPKKAKIVIP

(19) United States
(12) Patent Application Publication
(10) Pub. No.: US 2026/0218222 A1
(43) Pub. Date: Jul. 30, 2026

(54) CARBON SEQUESTRATION WITH TRANSGENIC PLANTS EXPRESSING CARBONIC ANHYDRASE

(71) Applicant: Wild Bioscience Ltd, Abingdon, Oxfordshire (GB)

(72) Inventors: Ross Hendron, Abingdon, Oxfordshire (GB); Steven Kelly, Abingdon, Oxfordshire (GB); Rona Costello, Abingdon, Oxfordshire (GB)

(21) Appl. No.: 19/145,241

(22) PCT Filed: Jan. 5, 2024
(86) PCT No.: PCT/GB2024/050026

§ 371 Date: Jul. 2, 2025

(30) Foreign Application Priority Data
Jan. 9, 2023 (GB) 2300297.5

(51) Int. Cl. C12N 15/82; C12N 9/88

(52) U.S. Cl. CPC C12N 15/8251; C12N 9/88; C12N 15/8227; C12Y 402/01001

(57) ABSTRACT

Root-specific expression of carbonic anhydrase increases the environmental carbon sequestration ability of plants. Here, a plant is genetically engineered so that its heritable genetic material comprises a gene encoding a transportable carbonic anhydrase protein under the control of a promoter active in root cells, such that the carbonic anhydrase is transported from the roots of the plant into the extracellular environment. These genetically altered plants demonstrate increased levels of carbonic anhydrase activity in the extracellular environment of the plant roots, which enhances carbon sequestration in extracellular environments such as soil.

### FIELD OF THE INVENTION

The present invention relates to the field of biotechnology, and more specifically to compositions and methods for enhancing sequestration of atmospheric CO₂ in soils using plants. Genetically modified plants expressing enzymes to facilitate CO₂ sequestration are provided, along with enzyme sequences and their use in various applications. The invention further concerns parts of such modified plants, such as plant cells, plant parts, plant organs, fruits, seeds, embryos, germplasm and processed plant products.

### BACKGROUND

Anthropogenic greenhouse gas emissions have already caused an approximately 1° C. rise in global surface temperature over pre-industrial levels (Masson-Delmotte, 2018). Carbon dioxide (CO₂) emissions have been the main contributor to this warming and efforts to meet the requirements of the Paris agreement to stay below 2° C., and ideally 1.5° C., to mitigate against the worst effects of climate change (Horowitz, 2016) will require the combined effort of reducing global CO₂ emissions and the development of technologies that increase the sequestration of atmospheric CO₂ (Masson-Delmotte, 2018).

### SUMMARY OF INVENTION

The inventors have unexpectedly found that carbonic anhydrase genes expressed in root cells can enable CO₂ sequestration, for example as bicarbonate, in the extracellular environment of the roots. In doing so, they have provided a solution to sequestration of atmospheric CO₂ that is relevant for industrial biotechnology. Prior to the present invention, carbonic anhydrase genes have not been over-expressed in plant root cells, nor have carbonic anhydrase proteins been exported from plant root cells, nor have carbonic anhydrase proteins been over-expressed on the surface of plant root cells or in the extracellular environment. Thus, the approach used herein is novel. Overall, the present invention achieves enhanced carbon sequestration from the atmosphere with no observable negative or deleterious anatomical, physiological, biochemical or developmental effects on modified plants.

The present invention provides a plant which comprises, or whose heritable genetic material comprises, a gene encoding a secretable/transportable carbonic anhydrase protein under the control of a promoter active in root cells, such that the carbonic anhydrase is secreted/transported from the roots of the plant into the extracellular environment. Therefore, the invention provides a plant in which the genetic material has been modified, the modification being such that the modified plants express carbonic anhydrase or a portion thereof, in at least some root cells of the plant. This expression in root cells is additional to any endogenous expression of carbonic anhydrase in the plant, in particular in the plant root where endogenous expression does not provide secretable/transportable carbonic anhydrase or any substantial carbonic anhydrase enzymatic activity in the extracellular environment of the plant root.

The expressed carbonic anhydrase may lead to increased levels of extracellular root carbonic anhydrase enzymatic activity, or in other words carbonic anhydrase enzymatic activity in the extracellular environment of the roots of the plant. The extracellular root environment may, for example, be the soil or any other location where a plant may germinate or grow or be cultivated including aeroponic and/or hydroponic growth environments.

Also provided herein is a plant wherein the carbonic anhydrase is over-expressed in the extracellular environment of the root cells in comparison to a plant which does not comprise, or whose heritable genetic material does not comprise, a gene encoding a secretable/transportable carbonic anhydrase protein under the control of a promoter active in root cells.

In addition or alternatively, provided herein is a plant wherein the heritable genetic material of the plant has been modified to contain a gene encoding a secretable/transportable carbonic anhydrase protein under the control of a promoter active in root cells. The genetic modification results in a carbonic anhydrase that is over-expressed in the extracellular environment of the plant root in comparison to a plant whose heritable genetic material does not comprise a gene encoding a transportable carbonic anhydrase protein under the control of a promoter active in root cells or has not been altered.

In some embodiments, the gene encoding the secretable/transportable carbonic anhydrase protein further comprises a signal peptide sequence for directing secretion/transport of the carbonic anhydrase into the extracellular environment of the plant root.

Also provided herein is a polynucleotide sequence comprising a sequence coding for a polypeptide sequence under the control of a suitable promoter, wherein the polypeptide sequence comprises a carbonic anhydrase sequence and a signal peptide sequence, wherein the promoter is active in plant roots and capable of driving expression of the carbonic anhydrase in root cells, wherein the signal peptide sequence is capable of directing secretion/transport of the carbonic anhydrase into the extracellular environment of a plant root. The signal peptide sequence of any polynucleotide sequence disclosed herein may either be natively encoded or translationally fused to the carbonic anhydrase sequence to ensure secretion/transport of the carbonic anhydrase to the extracellular environment of the plant root. The signal peptide sequence for directing secretion/transport may be derived from any secreted protein that is transported to the extracellular environment by a protein secretion pathway. The signal peptide sequence may also be derived from the extensin protein, and in some embodiments it may be derived from a carrot plant or derived from the vacuolar sorting protein, or derived from a pea plant.

The carbonic anhydrase (including the polynucleotide encoding it or the peptide itself) is transportable and, in some embodiments, may be secreted or secretable. Additionally, in some embodiments the carbonic anhydrase may be attached to the outside of the cell or to the plasma membrane by a transmembrane domain, or a transmembrane helix, or a membrane anchor, or a glycosylphosphatidylinositol (GPI) anchor, or any other biomolecule which causes the carbonic anhydrase to remain attached to the plasma membrane or cell wall following secretion from the cell.

The promoter, signal peptide sequence, or carbonic anhydrase of the polynucleotide or plant disclosed herein may be derived from a gene from the same plant species or variety as the plant in which it is expressed. The promoter may be root-specific, and in some embodiments it may be root hair and/or root epidermis specific. The expression domain of the promoter may also include other cells and tissues of the plant so long as the expression domain includes the root.

Also provided herein are carbonic anhydrases further comprising a transmembrane domain or a membrane anchor sequence. The carbonic anhydrase may be an α, β, γ, δ, ζ, η, θ, or ι subtype carbonic anhydrase. In some embodiments it is a monomer.

Provided herein are polypeptide sequences encoded by the polynucleotide disclosed herein. A plant cell, cell line or progeny thereof comprising the polynucleotide sequence or polypeptide sequence disclosed herein are also provided. The progeny may be a progeny of the plant cell or a progeny of the cell line.

In some embodiments, the plant, polynucleotide, polypeptide, cell, cell line or progeny disclosed herein may derive from a plant wherein the plant is a crop. In some embodiments the plant is a row or cover crop. In some embodiments, the plant is selected from a corn, soybean, pea, cotton, canola, camelina, potato, tomato, sugar beet, cassava, sweet potato, alfalfa, wheat, barley, sorghum, oat, sorghum, millet, rye, teff, rice, or clover, cress, brassicas, vetch and prairie grasses. Additionally, the polynucleotide, polypeptide, cell, cell line or progeny disclosed herein may derive from a plant wherein the plant is a tree. In some embodiments wherein the tree is poplar, spruce, pine, eucalyptus, oil palm or rubber.

Also provided herein is the plant, polypeptide, polynucleotide, cell, cell line or progeny disclosed herein, where more carbon is sequestered into the extracellular environment of the plant in comparison to a plant whose heritable genetic material does not comprise a gene encoding a transportable carbonic anhydrase protein under the control of a promoter active in root cells.

Additionally provided is a plant part, plant tissue, plant organ, plant cell, plant protoplast, embryo, callus, cell culture, pollen grain or seed, derived or obtained from the plant disclosed herein.

Provided herein is a vector comprising the polynucleotide sequence disclosed herein. The vector is optionally a plasmid. Plasmids in accordance with the invention may further comprise one or more other elements selected from: an enhancer, a plant selectable marker, a multicloning site, or a recombination site. The structure, modification, propagation and generation of vectors or plasmids is well known to a person of ordinary skill in the art.

Also provided are compositions for transformation of plant cells comprising the polynucleotide, vector, or plasmid disclosed herein, optionally comprising microparticles coated with said polynucleotide or said vector. The microparticles may be of metal or synthetic material. In some embodiments the metal is tungsten or gold.

Also provided is a bacterium comprising the polynucleotide, plasmid, or vector disclosed herein, optionally wherein the bacterium is E coli. or Agrobacterium sp. In some embodiments the agrobacterium is A. tumefaciens.

Also provided herein are plants comprising the polynucleotide disclosed herein stably integrated into the genome thereof; and in some embodiments heritably integrated into the genome thereof.

Provided herein are methods of producing a plant or seed, comprising the steps of:—
(a) providing the vector or plasmid disclosed herein;
(b) activating the promoter operably linked to the polypeptide coding DNA;
(c) optionally wherein the polypeptide is overexpressed in the extracellular environment of the cell in comparison to a plant whose heritable genetic material does not comprise a gene encoding a transportable carbonic anhydrase protein under the control of a promoter active in root cells.

Methods for increasing the capacity of a plant to sequester carbon in the soil are also provided herein, the methods comprising altering the heritable genetic material of the plant such that a carbonic anhydrase protein is transported from the roots of the plant into the extracellular environment. The altering of heritable genetic material may comprise inserting at least one polynucleotide into the heritable genetic material of a plant cell. The altering of heritable genetic material may also comprise genetically editing the heritable genetic material of a plant cell.

A method in accordance with the invention may employ classical and well-known techniques of genetic modification, involving a method of transformation, whereby the polynucleotide of the invention is incorporated into a plant genome. Any necessary root cell expression regulatory elements may be present. In some embodiments this incorporation is stable and heritable so as to permit introduction of the modification into particular lines of crop plants; advantageously for the purposes of crop improvement or breeding programmes. A gene editing method may be used to incorporate or give rise to the polynucleotide of the invention.

Although various methods for gene editing are known, including TALE Nucleases (TALENs) or Zinc Fingers, a CRISPR system may also be used. For example a CRISPR-Cas system may be used whereby a guide RNA (gRNA) is selected to target the action of a CRISPR-Cas protein to a desired genomic locus resulting in a homologous recombination (HR) event or non-homologous end joining repair, i.e. insertion-deletion of the polynucleotide into the plant genome.

Therefore, the polynucleotide disclosed herein may include a polynucleotide encoding a CRISPR-Cas protein, optionally also a guide RNA (gRNA), wherein the gRNA directs the CRISPR-Cas protein to the locus of an endogenous carbonic anhydrase coding sequence in the plant cell genome, optionally wherein a regulatory element is inserted so as to cause expression of the carbonic anhydrase in at least some root cells of the plant.

Also provided herein are processed plant products obtained from the plant or the plant part, plant tissue, plant organ, plant cell, plant protoplast, embryo, callus culture, pollen grain or seed disclosed herein; optionally wherein the processed product comprises a detectable nucleic acid sequence of (i) a transportable carbonic anhydrase downstream of a gene expression regulatory element active in at least some of the root cells of a plant, or (ii) a polynucleotide or at least a portion of a polynucleotide disclosed herein.

### BRIEF DESCRIPTION OF THE FIGURES

The invention will be further described by way of reference to the examples and accompanying figures.

FIG. 1 depicts an illustrative diagram of the genetic vector used for soybean transformation to express a secreted-version of a soybean carbonic anhydrase in roots.

FIG. 2 demonstrates that the carbonic anhydrase activity of the extracellular environment around the roots is enhanced in genetically engineered plants compared to wild type (control plants). A) shows carbonic anhydrase activity in a supernatant following enzyme liberation from root tissue of control and engineered soybean hairy roots. Standard error bars shown. B) shows carbonic anhydrase activity in the hydroponic solution in which control and genetically engineered plants were grown. Standard error bars shown. C) shows that genetically engineered plants produce a measurable decrease in soil pH relative to both soil-only control pots and control plants. This demonstrates that carbonic anhydrase has catalysed the conversion of CO₂ in the air into bicarbonate and a proton in the soil. Standard error bars shown. D) shows that engineered plant roots sequester CO₂ from the atmosphere, which control plant roots are not able to do. CO₂ sequestration is shown for pots containing soil and plant roots (above ground tissue excised) over a 30-minute measurement period. Standard error bars are shown. E) shows carbonic anhydrase activity in the extracellular environment of the roots of wild type and transgenic plants. This shows that transgenic plants have higher carbonic anhydrase activity. F) shows that there are no discernible differences in growth phenotype between control and genetically engineered Arabidopsis plants that secrete carbonic anhydrase from roots. Photo taken of 5-week-old control plant (left) and engineered plant of this invention (right). Scale bar represents 5 cm.

FIG. 3 depicts a general schematic for the DNA sequence of the fusion carbonic anhydrase protein. Such constructs are typified by the inclusion of a genetic sequence encoding a protein with carbonic anhydrase activity, a promoter ensuring the sequence is expressed e.g. in plant root tissues, a terminator, and a peptide sequence designating cellular export e.g. an N-terminus signal peptide.

FIG. 4 demonstrates that the carbonic anhydrase activity of the extracellular environment around the roots is enhanced in genetically engineered plants compared to wild type (control plants). A) shows that carbonic anhydrase activity in the hydroponic solution in which genetically engineered soybean plants that transport carbonic anhydrase from the roots of the plant into the extracellular environment were grown is higher relative to control plants. Standard error bars shown. B) shows that genetically engineered soybean plants that transport carbonic anhydrase from the roots of the plant into the extracellular environment produce a measurable decrease in soil pH relative to control plants. This demonstrates that carbonic anhydrase has catalysed the conversion of CO₂ in the air into bicarbonate and a proton in the soil. Standard error bars shown. C) shows that genetically engineered soybean plants with carbonic anhydrase embedded on root surface produce a measurable decrease in soil pH relative to control plants. This demonstrates that carbonic anhydrase has catalysed the conversion of CO₂ in the air into bicarbonate and a proton in the soil. Standard error bars shown. D) shows increased carbonic anhydrase activity in the hydroponic solution in which genetically engineered soybean plants with an alternative carbonic anhydrase were grown, relative to hydroponic solution in which control plants were grown. Standard error bars shown. E) demonstrates increased carbonic anhydrase activity from the roots of genetically engineered soybean plants relative to control plants. Standard error bars shown. F) shows that there are no discernible differences in growth phenotype between control and genetically engineered soybean plants that secrete carbonic anhydrase from roots. Photo taken of 5-week-old control plant (left) and engineered plant of this invention (right). Scale bar represents 5 cm.

FIG. 5A+B: Confocal images of soybean protoplasts expressing fluorescent protein tagged carbonic anhydrase protein. A) Conticribra weissflogii carbonic anhydrase fused with a fluorescent protein. B) Conticribra weissflogii carbonic anhydrase fused with a fluorescent protein with the PsBP80 signal peptide at the N-terminus of the fusion protein and the PsBP80 transmembrane domain at the C-terminus of the fusion protein. C+D: Light microscope images of GUS stained Arabidopsis thaliana roots. C) GUS staining of Arabidopsis roots expressing GUS under the control of the pGmPIN2b promoter. D) GUS stain of Arabidopsis roots expressing GUS under the control of the pGmEXPA7 promoter. Scale bar represents: A) 10 μm, B) 20 μm, C) 50 μm, D) 50 μm. Arrows on A) highlight cytoplasmic GFP expression and B) highlight fluorescent protein expression localised to plasma membrane of the soybean protoplast. Arrows on C) left image and right image and D) left image, highlight areas of GUS stained root.

FIG. 6 demonstrates carbonic anhydrase activity of the extracellular environment around the roots in genetically engineered plants enhanced inorganic carbon sequestration in soil compared to wild type (control plants) A) Analysis of soil inorganic carbon content demonstrating that the soil in which engineered soybean plants of this invention are grown accumulates more inorganic carbon compared to soil in which control soybean plants are grown and no plants are grown. B) shows that following treatment with hydrated lime Ca(OH)₂, soil in which engineered Arabidopsis thaliana plants of the invention are grown accumulates more soil inorganic carbon than soil in which control Arabidopsis thaliana plants are grown. C) shows that the result shown in B is consistent when soil inorganic carbon content is normalised by soil organic carbon content. D) shows that soil in which engineered soybean plants of this invention are grown accumulates more soil inorganic carbon than soil in which control soybean plants are grown. E) shows that the result shown in D is consistent when soil inorganic carbon content is normalised by soil organic carbon content.

### DETAILED DESCRIPTION

### Weathering Mediated CO₂ Sequestration

One way that CO₂ is naturally sequestered from the atmosphere is through the process of chemical rock weathering. This occurs when atmospheric CO₂ dissolves in rainwater to form carbonic acid (H₂CO₃). When carbonic acid encounters carbonate and/or silicate rock a chemical reaction occurs that causes the rock to breakdown and ions of bicarbonate (HCO₃⁻) and calcium (Ca²⁺) to be released. These ions are carried by surface waters to the ocean where they precipitate out and fall to the ocean floor for long-term burial (i.e. hundreds of thousands of years). The ability of this process to facilitate long-term sequestration of atmospheric CO₂ has led to the proposal of methods that accelerate the chemical rock weathering. These methods, known as enhanced weathering, involve substantially increasing the surface area of rock exposed to rain (containing dissolved CO₂) by mechanically crushing it into dust and spreading it over terrestrial ecosystems. This has been proposed to greatly increases the rate at which rock is weathered, and thus the rate at which CO₂ is sequestered from the atmosphere (Strefler et al., 2018).

### Carbonic Anhydrase

Carbonic anhydrases are a group of naturally occurring enzymes that catalyse the interconversion of gaseous carbon dioxide (CO₂) and water (H₂O) into ions of bicarbonate (HCO₃⁻) and hydrogen (H⁺) [CO₂+H₂O⇄HCO₃⁻+H⁺]. Carbonic anhydrase is a widely distributed enzyme, with members of this enzyme family found in organisms from across the tree of life, from bacteria and archaea to plants and animals. Given the activity of the enzyme there has been interest in using carbonic anhydrase enzymes in industrial settings to capture CO₂ from industrial flue gas (as discussed in Boone et al., 2013 and Steger et al., 2022). WO 2015183935A2 also describes the use of modified carbonic anhydrases to catalyse the removal of CO₂ from industrial flue gas. Also disclosed is the use of modified carbonic anhydrases which are affixed to a matrix and cross-linked with mineral ions so that a bicarbonate salt is precipitated out, thereby removing the CO₂. This process is used in desalination and extracorporeal CO₂ removal. Similarly, WO2013159228A1 describes an industrial process whereby CO₂ is contacted with a carbonic anhydrase solution containing an additional absorption compound which together sequester the CO₂.

Carbonic anhydrases (CAs) are metalloenzymes that contain a metal co-factor (typically Zn²⁺ but in some cases Cd²⁺, Co²⁺, Fe²⁺ and Mn²⁺ although one newly described class of carbonic anhydrase has been shown to exhibit activity under metal-free conditions (Hirakawa et al., 2021)). To date, eight classes of carbonic anhydrases have been identified (α, β, γ, δ, ζ, η, θ, and ι) with varying distribution across the three domains of life. α, β, and γ carbonic anhydrases are present in eukaryotes and prokaryotes, while other classes are found in some species of microalgae and protists. A recently described 1-CA has been identified in the microalgae Thalassiosira pseudonana and the gram-negative bacterium Burkholderia territorii. Depending on the class, carbonic anhydrases may be active as monomers, dimers, trimers, tetramers, or octomers. In addition to their diversity in form, carbonic anhydrases have diverse functions between and within organisms. In plants, for example, carbonic anhydrases are present in several different plants tissues and vary in their subcellular localisation (DiMario et al., 2017). All carbonic anhydrases, irrespective of their class or the organism in which they are found, catalyse the same biochemical reaction, i.e. the interconversion of gaseous CO₂ and water (H₂O) into ions of bicarbonate (HCO₃⁻) and hydrogen ions (H⁺) as described above.

Recently, Matt et al. 2022 have described the expression of a glycosylphosphatidylinositol-anchored membrane-bound extracellular carbonic anhydrase in the calcifying primary mesenchyme cells of sea urchin larva. Extracellular CO₂ hydration is demonstrated in these findings to suggest the resulting bicarbonate is taken up by the cell for the construction of carbonate structures that form the skeletal tissue of this organism.

Plants present an interesting opportunity for harnessing carbonic anhydrase for CO₂ sequestration as bicarbonate in the soil and for the long-term sequestration of that bicarbonate via ground waters in an analogous manner to the chemical weathering process. Plant roots are in direct contact with the soil and with gaseous CO₂ in the soil, which is itself in equilibrium with atmospheric CO₂.

Historically, plants have been genetically engineered to express and secrete enzymes. Li et al. 2009 describes soybean plants which have been transformed with a construct containing a gene encoding a phytase from the fungus Aspergillus ficuum fused at the N-terminus with a signal peptide sequence from a carrot extensin gene. This recombinant gene was then fused to a promoter from the Arabidopsis Pky10 gene which conferred gene expression in the roots of the transgenic soybean plants.

Here we propose engineering plants to express in their root cells (specifically the outer cell layer, i.e. epidermal cells) an engineered form of carbonic anhydrase which is secreted from the cell into the extracellular environment. In particular, targeting plants which cover a large surface area of the globe (e.g., crop plant species) would provide a means by which atmospheric CO₂ could be sequestered at large scale as bicarbonate ions in the soil. Following transport by surface waters, some of this bicarbonate is expected to be carried to, and buried in, the oceans.

Bioremediation denotes any process which employs a biological system (such as plants or bacteria) to remove environmental pollutants such as CO₂ from natural or industrial settings. Bioremediation methods, such as that presented by the present invention, have particular advantages. Bioremediation often requires fewer resources and less energy than industrial man-made technologies and can be specifically altered through genetic engineering to address the specific needs of a particular environment. Additionally, these biological methods do not produce hazardous waste as by-products, and therefore do not require expensive specialist waste disposal.

A need exists in the art to find ways to accelerate long-term CO₂ sequestration by utilising bioremediation methods. Moreover, there is a need to achieve this through scalable methods that can be implemented rapidly and globally to urgently address the issue of anthropogenic CO₂ release and elevated atmospheric CO₂ concentration. The present invention addresses this need by demonstrating that expression of carbonic anhydrase in roots of plants, leading to increased levels of extracellular carbonic anhydrase enzymatic activity, results in enhanced CO₂ sequestration through conversion of CO₂ to bicarbonate. The present invention also demonstrates that multiple different natural or engineered carbonic anhydrase genes from different species can provide this function in plants. Unexpectedly, the inventors have found that this function can be provided when carbonic anhydrase proteins are secreted from plants, and/or when carbonic anhydrase proteins are anchored to the external periphery of the cell, either through for example a transmembrane domain or an anchor. Optionally, the N-terminus or C-terminus may be fused to a transmembrane domain, transmembrane helix, or a glycosylphosphatidylinositol (GPI) anchor, or other biomolecule which causes the carbonic anhydrase to remain attached to the plasma membrane or cell wall following secretion from the cell. The present invention also demonstrates that carbonic anhydrase proteins in various compositional forms described above can be expressed in plants using a variety of promoters that achieve the desired expression in root tissue.

The following detailed description conveys exemplary embodiments of the present invention in sufficient detail to enable those of ordinary skill in the art to practice the present invention. Features, or limitations of the various embodiments described do not necessarily limit other embodiments of the present invention, or the present invention as a whole. Hence the following detailed description does not limit the scope of the present invention, which is defined only by the claims.

Conventional techniques in botany, microbiology, tissue culture, molecular biology, chemistry, biochemistry and recombinant DNA technology and bioinformatics for use in employing the present invention are all readily known and available to a person of average skill in the art. Specific techniques are explained fully in the literature.

In the present specification the term “genetically engineered’ is used and as will be familiar to a person of skill in the art this term may encompass the terms “genetically modified” and “gene editing”, each of which is appreciated in the art as having respectively differing meanings and underlying technical methodology. However, the term “modified” in an unqualified sense herein, may be understood more simply to mean “changed”, “altered” or “different”. The terms “altered”, “changed” and “modified” may be used interchangeably herein. The terms “increase”, “improve” or “enhance” are used interchangeably herein.

The present invention provides a plant whose heritable genetic material comprises a gene encoding a transportable carbonic anhydrase protein under the control of a promoter active in root cells, such that the carbonic anhydrase is transported from the roots of the plant into the extracellular environment.

The plant of the invention may be a genetically modified plant, a genetically enhanced plant, a non-naturally occurring plant and/or a genetically engineered plant. In some instances, the plant may be transgenic plant.

Plants in accordance with the invention may be monocots or dicots; in some embodiments they are crop plants, e.g. fruits, vegetables, cereals, oilseed, and legumes, and may commonly be used for food, animal feed, biofuel or biomass production. A crop plant is any plant which is grown on a commercial scale for human or animal consumption or use. The plants may also be trees, particularly those popular in forestry and land management, carbon capture and biofuel or biomass production. The plants may be horticultural plants. In some embodiments, the plants are cover crops which are plants used to cover exposed soil between harvesting. Cover crops are utilised to mitigate soil erosion, increase soil fertility and quality, in the conservation of water, management of weeds/pests and diseases and to maintain biodiversity and wildlife in an agroecosystem. Cover crops may be utilized during a fallow period in arable farming. During a fallow period, arable land is not sown with harvestable seed for at least one or more vegetative cycles. This allows the arable land to recover. Cover crops may be sown during a fallow period to assist in the recovery of the land.

Heritable genetic material denotes any polynucleotide sequence and any associated genetic elements in any cell or organelle which is capable of being passed from one generation to a subsequent generation through sexual or asexual reproduction. Sexual plant breeding methods may include propagation. Asexual reproduction, also known as clonal propagation, by way of grafting plant cuttings is also considered as the incorporation of heritable genetic material into the genome of the plant. The heritable genetic material may therefore only be present in part of the plant.

The term “gene” denotes a polynucleotide sequence which codes for a polypeptide in full or in part comprising introns and exons, and an open reading frame (ORF) to enable transcription.

The term code denotes the ability of a nucleotide sequence to act as a template for translation, whereby the arrangement of nucleotides represents codons which further represent amino acids. A gene may further comprise additional genetic elements such as a promoter, repressor, terminator, enhancer and/or transcription factor binding elements. A gene may be inserted into the genome of an organism using genetic engineering methods known in the art, such as transformation. A gene may be exogenous (i.e. not native, inserted into the existing genome) or endogenous (i.e. exists within the genome as a native gene).

The polypeptide of the invention may be a fusion protein. A fusion protein is a polypeptide which derives from a single nucleotide sequence which contains two or more genes or portions of genes, wherein the genes code for separate polypeptides which would not natively be transcribed and translated together.

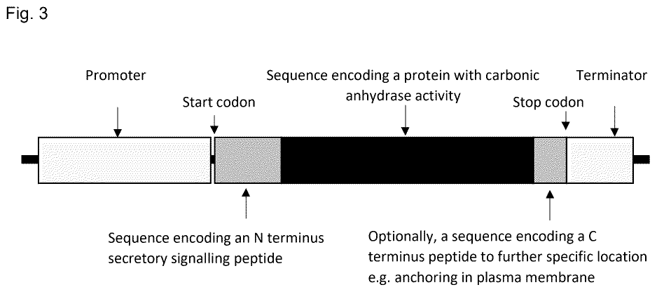

Typically, the polynucleotide of the invention comprises a promoter, a start codon, a sequence encoding an N terminal signal peptide, a sequence encoding a protein with carbonic anhydrase activity, optionally a sequence encoding a C terminus peptide which targets the polypeptide to a further specific location e.g. anchoring in the plasma membrane, a stop codon and a terminator, as typified in FIG. 3.

The coding sequence of the carbonic anhydrase encoded in the DNA polynucleotide sequence of the invention may correspond to any of the polynucleotide sequences of SEQ ID NO: 1, or SEQ ID NO: 2, or a sequence of at least 65% identity with any of said sequences; or a sequence of at least 70% identity with any of said sequences; or a sequence of at least 80% identity with any of said sequences, or at least 85%, at least 90%, or at least 95%, identity with any of said sequences. In some embodiments, the carbonic anhydrase will have any of the % homologies referred to herein and retain the carbonic anhydrase enzymatic activity.

The amino acid sequence of the carbonic anhydrase encoded in the polynucleotide sequence(s) of the invention may correspond to any of the amino acid sequences of SEQ ID NO: 8, or SEQ ID NO: 9, or a sequence of at least 65% identity with any of said sequences; or a sequence of at least 70% identity with any of said sequences; or a sequence of at least 80% identity with any of said sequences, or at least 85%, at least 90%, or at least 95%, identity with any of said sequences. In some embodiments, the carbonic anhydrase will have any of the % homologies referred to herein and retain the carbonic anhydrase enzymatic activity, preferably in the extracellular root environment.

The polynucleotide may correspond to a full-length carbonic anhydrase, or a portion thereof. The term “portion”, “variant”, “homolog” or other representative wording in the art as used herein used in relation to a carbonic anhydrase sequence, or a functional fragment thereof, means any carbonic anhydrase ortholog of a differing polynucleotide or polypeptide sequence from any species, including plants.

In terms of percentage identity to a reference sequence, such as SEQ ID NO: 1, 2, 8 or 9 a variant of a carbonic anhydrase may have, in increasing order of preference, at least 65%, at least 66%, at least 67%, at least 68%, at least 69%, at least 70%, at least 71%, at least 72%, at least at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% overall sequence identity to that reference sequence.

Carbonic anhydrases are enzymes which catalyse the reversible conversion of water (H₂O) and carbon dioxide (CO₂) into bicarbonate (HCO₃⁻) and hydrogen (H⁺), as detailed below:—

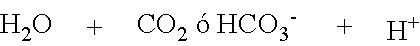

In the absence of carbonic anhydrase enzyme activity the pH of the aqueous environment determines whether the forward or backward reaction is preferred. Carbonic anhydrases are widely distributed in all organisms. Carbonic anhydrases alter the rate at which this reaction occurs and the equilibrium between CO₂ and HCO₃⁻. Carbonic anhydrases from different species may have minimal sequence similarity but can adopt similar tertiary structures to catalyse this reaction in very different contexts.

In some embodiments, the carbonic anhydrase may be an α, β, γ, δ, ζ, η, θ, or ι subtype carbonic anhydrase, and in some embodiments it is a monomer. Three classes of carbonic anhydrases natively exist in plants; α, β and γ subtypes. The β subtypes are better characterised and include isoforms with high efficiency for CO₂ hydration; for example, β subtypes function as part of C₄ photosynthesis in C₄ plants. β subtypes are dimeric, whilst a subtypes are monomeric. Monomeric subtypes have the advantage that there is no requirement for multimer formation at the surface of the cell.

The carbonic anhydrase sequence used in this invention may be a homolog, analog, ortholog, or paralog of any subtype of carbonic anhydrase isolated from any species. It would be expected by a person of ordinary skill in the art that any carbonic anhydrase enzyme would work equivalently in this invention, so long as it was suitable for expression by the plants of the invention (for example it consisted of naturally occurring amino acids in protein form). The Examples of the present application demonstrate that sequence identity or similarity between carbonic anhydrases is not required in order for plants of the invention to exhibit their surprising ability to sequester CO₂. As noted above, carbonic anhydrases from different species can adopt similar tertiary structures regardless of sequence similarity. The skilled person would therefore understand that a plant of the invention overexpressing any type of carbonic anhydrase in the extracellular environment of the root cells would function to sequester CO₂. The skilled person understands carbonic anhydrases are defined by their function. As such, any enzyme that catalyses the interconversion of gaseous CO₂ and water (H₂O) into ions of bicarbonate (HCO₃⁻) and hydrogen ions (H⁺) is envisaged as a carbonic anhydrase protein as part of the invention.

In some embodiments, a carbonic anhydrase with high catalytic efficiency in the direction of bicarbonate formation is selected, such that the reverse reaction would not be favored. A carbonic anhydrase may be selected based on efficacy, for example, determined via an enzyme activity assay. In addition, codon usage may be altered to enhance expression for a particular plant species.

In some embodiments, in any aspect of the invention herein defined, the promoter is a root cell specific promoter (i.e. a root cell specific promoter, or a promoter that is expressed in root cells). A promoter active in root cells is a promoter which is presently enabling expression of a polynucleotide sequence in a root cell, i.e. it is sufficient for protein production in root cells. The promoter may be active in other cells of the plant, and in some embodiments comparatively greater in root cells. The activity of the promoter may be enhanced when compared to a wild-type promoter. The activity of the promoter may be conditionally enhanced. The promoter may be an inducible promoter. The activity of the promoter may vary, such as seasonally, diurnally or in response to an external signal. i.e. temperature, pH, nutrient content of the soil. The promoter may be constitutively active.

The promoter may be a synthetic promoter comprised of various selected elements. For example, such a synthetic promoter may comprise root cell specific transcription factor binding element upstream of a promoter element. There may be two or more transcription factor binding elements which may be the same or different. A plurality of such transcription factor binding elements may serve to enhance the activity and/or specificity of the promoter in root cells. The promoter may be derived from non-plant organisms, such as the 35S promoter.

In other aspects, the root cell specific promoter may be derived from a gene that is expressed preferentially or specifically in root cell of plants and therefore is a naturally occurring promoter. The gene may be expressed in other plant cell types at endogenous levels. The promoter may be selected based on its predicted function in a particular species. A person of skill in the art is well aware of many root cell specific promoters. The root cell specific promoter may be active in some or all other plant cells at some expression level but be highly expressed in the roots.

The root specific promoter encoded in the DNA polynucleotide sequence of the invention may correspond to any of the polynucleotide sequences of SEQ ID NO: 6, or SEQ ID NO: 7, or a sequence of at least 65% identity with any of said sequences; in some embodiments a sequence of at least 70% identity with any of said sequences; and in some embodiments a sequence of at least 80% identity with any of said sequences, or at least 85%, at least 90%, at least 95%, identity with any of said sequences. In some embodiments, the encoded carbonic anhydrase will have any of the % homologies referred to herein and retain the carbonic anhydrase enzymatic activity.

The polynucleotide may correspond to a full-length root specific promoter, or a portion thereof. As mentioned above, it would be expected by a person of ordinary skill in the art that any promoter active in the roots of plants would work equivalently in this invention.

The term “portion”, “variant”, “homolog” or other representative wording in the art as used herein used in relation to a root specific promoter sequence, or a functional fragment thereof, means any root specific promoter ortholog of a differing polynucleotide or polypeptide sequence from any species, including plants.

In terms of percentage identity to a reference sequence, such as SEQ ID NO:6 or 7, a variant of a root specific promoter may have, in increasing order of preference, at least 65%, at least 66%, at least 67%, at least 68%, at least 69%, at least 70%, at least 71%, at least 72%, at least at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% overall sequence identity to that reference sequence.

In one embodiment a plant whose heritable genetic material comprises a gene encoding a transportable carbonic anhydrase protein under the control of a promoter active in root cells is provided wherein the gene encoding a transportable carbonic anhydrase protein comprises SEQ ID NO: 1 or SEQ ID NO: 2 or a sequence having at least 65% identity to SEQ ID NO: 1 or SEQ ID NO: 2, optionally wherein the promotor comprises SEQ ID NO: 6 or SEQ ID NO: 7 or a sequence having at least 65% identity to SEQ ID NO: 6 or SEQ ID NO: 7.

In one embodiment a plant whose heritable genetic material comprises a gene encoding a transportable carbonic anhydrase protein under the control of a promoter active in root cells is provided wherein the gene encoding a transportable carbonic anhydrase protein comprises SEQ ID NO: 1 or SEQ ID NO: 2 or a sequence having at least 65% identity to SEQ ID NO: 1 or SEQ ID NO: 2, optionally wherein the promotor comprises SEQ ID NO: 6 or SEQ ID NO: 7 or a sequence having at least 65% identity to SEQ ID NO: 6 or SEQ ID NO: 7; and wherein the signal peptide sequence comprises SEQ ID NO: 3 or SEQ ID NO: 4 or a sequence having at least 65% identity to SEQ ID NO: 3 or SEQ ID NO: 4.

The root cell specific promoter may be ubiquitously active in all plant cells at endogenous levels. Endogenous levels of carbonic anhydrase mRNA and full-length polypeptide are lower in cells which are not root cells. The expression level of carbonic anhydrase may exceed endogenous levels in any biological context whereby carbonic anhydrase is expressed. The biological context may be in response to a change in the environment.

The term secreted is used to describe the transport of a protein from the inside to the outside of a cell. It is used interchangeably with transported or exported. Thus a secretable/transportable carbonic anhydrase is capable of being secreted/transported to the extracellular environment. In some embodiments, the carbonic anhydrase is transported across the plasma membrane via the secretory pathway. Secretory pathways can be conventional or non-conventional. In some embodiments, the carbonic anhydrase is transported through the conventional secretory pathway, resulting in transport of the carbonic anhydrase across the cell membrane at the cell periphery. Non-conventional transport of proteins such as carbonic anhydrase can occur through one of four pathways; direct protein translocation across the plasma membrane through membrane transport proteins, lysosomal secretion, exosome release and blebbing. Transport of the carbonic anhydrase may be active or passive. A secreted protein such as the carbonic anhydrase of the invention is in some embodiments located to the plasma membrane (also known as the extracellular membrane) in a cell. The carbonic anhydrase may be in contact with the extracellular membrane via an extracellular matrix protein such as a glycoprotein, such as extensin, or via a polysaccharide. Suitable glycoprotein and polysaccharide anchors are well known in the art. The carbonic anhydrase may be in contact with the plasma membrane via a molecular anchor such as a transmembrane domain, transmembrane helix, or a glycosylphosphatidylinositol (GPI) anchor, or other biomolecule which causes the carbonic anhydrase to remain attached to the plasma membrane or cell wall following secretion from the cell. The carbonic anhydrase may be in contact with the plasma membrane via part of the carbonic anhydrase polypeptide of the invention, wherein part of the polypeptide represents a transmembrane protein. There may be one or more transmembrane domains. The carbonic anhydrase may not be in contact with the plasma membrane. The secreted/transported carbonic anhydrase may be adjacent to the plasma membrane or within the extracellular environment in close proximity of the plasma membrane, such as within the cell wall region. The carbonic anhydrase may remain in close proximity to the plasma membrane, but beyond the cell wall region. In some embodiments, the carbonic anhydrase is secreted into the immediate environment surrounding the roots, such as soil or alternative bedding medium, when the plant is present in such a medium. In some embodiments, the carbonic anhydrase is not secreted within a plant nodule. The carbonic anhydrase may continue to be secreted/transported in ex vivo/ex planta when the plant is separated from another part of itself, i.e. a cutting. The secreted/transported carbonic anhydrase may diffuse out into the extracellular environment or remain on the root surface once secreted. The carbonic anhydrase secreted from the root cells may diffuse to other tissue or cell extracellular environments, such as those adjacent to root cells. This may include any cell in close proximity with the roots or the rhizophere.

The carbonic anhydrase may be released from the cell via a non-secretable pathway, such as apoptosis, or any physical interference with the cell membrane which exposes intracellular carbonic anhydrase to the extracellular environment.

The carbonic anhydrase may be over-expressed in the extracellular environment of the root cells in comparison to a plant whose heritable genetic material does not comprise a gene encoding a transportable carbonic anhydrase protein under the control of a promoter active in root cells.

The term “over-expressed” regarding carbonic anhydrase refers to the expression level of carbonic anhydrase protein produced in comparison to endogenous baseline expression. The term “produced” is understood to refer to the production of the carbonic anhydrase protein and may be used interchangeably with the term expressed. Plants whose heritable genetic material does not comprise a gene encoding a transportable carbonic anhydrase protein under the control of a promoter active in root cells will exhibit endogenous levels of carbonic anhydrase in all biological contexts and these levels of carbonic anhydrase will be used as a baseline comparison for the plants of the invention. Baseline levels of carbonic anhydrase typically reflect endogenous levels of carbonic anhydrases which may fluctuate according to the internal and external environment of a plant. Increased levels of carbonic anhydrase, i.e. any amount above baseline, will be considered to denote over-expression of the carbonic anhydrase protein. In some embodiments, the plant of the invention expresses 0.005%, 0.1%, 0.5%, 1%, 2%, 3%, 4%, 5%, 10%, 25%, 50%, 75%, 100% or 200% additional carbonic anhydrase protein in comparison to endogenous baseline expression. Endogenous baseline expression may be established in a control plant which may be a wild-type plant. Cells of the same type and morphology in the same location of the plant, i.e. root cells should be compared to one another.

A plant whose heritable genetic material does not comprise a gene encoding a transportable carbonic anhydrase protein under the control of a promoter active in root cells is a plant which lacks a gene in its genome which encodes for a transportable carbonic anhydrase protein under the control of a promoter which is active in root cells. This plant has not been modified in the same fashion as the plants of the invention. This plant may be known as an untransformed plant, compared to a transformed plant of the invention. The plant may comprise other genetically modified elements related to or unrelated to carbonic anhydrases which are distinct from those of the invention. A plant whose heritable genetic material does not comprise a gene encoding a transportable carbonic anhydrase protein under the control of a promoter active in root cells may be referred to or used as a “control” plant. Accordingly, the control plant has not been genetically modified to alter expression of the polynucleotide and/or polypeptide disclosed herein. This plant will exhibit endogenous levels of carbonic anhydrase in all biological contexts and these levels of carbonic anhydrase will be used as a baseline comparison for the plants of the invention. Baseline levels of carbonic anhydrase typically reflect endogenous levels of carbonic anhydrases which may fluctuate according to the internal and external environment of a plant. A control plant may be a wild-type plant. A control plant may be a wild-type plant of the same species as the plant of the invention. The control plant function is to provide a reliable reference against which the expression of polynucleotides or polypeptides of the invention can be compared against in a modified plant material. The plant of the invention may therefore be genetically engineered compared to a non-engineered plant. The control plant may be defined by determining the level of carbonic anhydrase activity by enzyme assays known in the art and defined herein. Carbonic anhydrase activity may be undetectable or at very low levels in a control plant when compared with the plant of the invention. A plant of the invention exhibits increased levels of carbonic anhydrase activity, i.e. any amount above of increased activity as compared to a control plant. Typically, a more than two-fold increase in carbonic anhydrase activity is observed in the plant of the invention compared to a control or unmodified plant. In some embodiments, the plant of the invention has more than a two-fold, three-fold, four-fold or five-fold increase in carbonic anhydrase activity compared to a control plant. In some embodiments, the plant of the invention has more than a 0.005%, 0.1%, 0.5%, 1%, 2%, 3%, 4%, 5%, 10%, 20%, 30%, 40%, 50%, 60%, 70%, 80%, 90% or 100% increase in carbonic anhydrase activity compared to a control plant. The carbonic anhydrase activity may be measured in the root cells, the whole root or whole plant. In some embodiments, the plant of the invention has more than a two-fold, three-fold, four-fold or five-fold increase in carbonic anhydrase activity in the extracellular space compared to a control plant. In some embodiments, the carbonic anhydrase activity is quantified using the Wilbur-Anderson Units (WA) equation, or carbonic anhydrase esterase activity measured by the rate to catalyze hydrolysis of p-Nitrophenol acetate (pNPA) to known concentration of p-Nitrophenol (pNP) per unit of time which can alternatively be expressed in enzyme activity units such as katal (kat) or international units (IU), or carbonic anhydrase activity measured by units of conductivity changed per unit time, or carbonic anhydrase activity quantified using radiotracer based assays where activity is reported in counts or disintegrations per unit of time, or other equivalent methods to quantify carbonic anhydrase activity which is well understood by the skilled person. Optionally, the plant of the invention has a two-fold, three-fold, four-fold or five-fold increase in carbonic anhydrase activity in WA units compared to a control plant.

The gene encoding the transportable carbonic anhydrase protein may further comprise a signal peptide sequence for directing transport of the carbonic anhydrase into the extracellular environment of the plant root. The term “signal peptide” may also be known as a signal sequence, targeting signal, localization signal, localization sequence, transit peptide, target peptide, leader sequence or leader peptide. The signal peptide is usually present at the N-terminal of a polypeptide but may be present at the C-terminus. In the present invention, the signal peptide is present at the N-terminus.

Membrane-bound proteins can be type I or II; type I membrane-bound proteins rely on a signal peptide which directs them towards the secretory pathway which is subsequently cleaved, whereas type II proteins are targeted to the secretory pathway via their first transmembrane domain, which is not cleaved. Therefore, the signal peptide of the invention may or may not include a transmembrane domain.

The signal peptide sequence encoded in the DNA polynucleotide sequence of the invention may correspond to any of the polynucleotide sequences of SEQ ID NO: 3, or SEQ ID NO: 4, or a sequence of at least 65% identity with any of said sequences; in some embodiments a sequence of at least 70% identity with any of said sequences; and in some embodiments a sequence of at least 80% identity with any of said sequences, or at least 85%, at least 90%, at least 95%, identity with any of said sequences. In some embodiments, the carbonic anhydrase encoded from the above DNA sequences will have any of the % homologies referred to herein and retain the carbonic anhydrase enzymatic activity.

The polynucleotide may correspond to a full length signal peptide, or a portion thereof.

The term “portion”, “variant”, “homolog” or other representative wording in the art as used herein used in relation to a signal peptide sequence, or a functional fragment thereof, means any signal peptide sequence of a differing polynucleotide or polypeptide sequence from any species, including plants.

In terms of percentage identity to a reference sequence, such as SEQ ID NO:3 or 4, a variant of a signal peptide sequence may have, in increasing order of preference, at least 65%, at least 66%, at least 67%, at least 68%, at least 69%, at least 70%, at least 71%, at least 72%, at least at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% overall sequence identity to that reference sequence.

The signal peptide sequence cooperates with the promoter to ensure extracellular expression and transport of the polypeptide sequence to the extracellular environment.

Also provided herein is a polynucleotide sequence comprising:
a suitable promoter;
a sequence coding for a polypeptide sequence under the control of said suitable promoter; and
a sequence encoding a signal peptide sequence;
   wherein the polypeptide sequence comprises a carbonic anhydrase sequence, and wherein the promoter is active in plant roots and capable of driving overexpression of the carbonic anhydrase, the signal peptide sequence being capable (prior to removal) of directing transport of the carbonic anhydrase into the extracellular environment of a plant root.

A suitable promoter is a promoter which enables the expression of a polypeptide sequence to which it is associated. The signal peptide sequence is sufficient to enable transport of the carbonic anhydrase into the extracellular environment of a plant root.

The signal peptide sequence may either be natively encoded or translationally fused to the carbonic anhydrase sequence to ensure transport of the carbonic anhydrase to the extracellular environment of the plant root.

The signal peptide sequence for directing transport may be derived from a secreted protein that is transported to the extracellular environment by a protein secretion pathway, and in some embodiments the conventional (or canonical) protein secretory pathway. The signal peptide sequence may be derived from the extensin protein, and in some embodiments it is derived from a carrot plant or derived from the vacuolar sorting protein, or it may be derived from a pea plant.

The plant or polynucleotide according to the invention may have a promoter, and/or a signal peptide sequence derived from a gene from the same plant species or variety as the plant. The plant or polynucleotide according to the invention may have a promoter and/or a signal peptide sequence derived from a gene from a different plant species, cultivar or variety as the plant. Plant varieties are genetic variations of the same species of plant which arise naturally in the environment. Cultivars are genetic variations of the same species of plant which have been develop by humans through controlled breeding.

The promoter sequence, signal peptide sequence, transmembrane sequence and any other sequences present in the polynucleotide may be derived from any species, variety or cultivar.

The promoter of the invention is active in plant tissues that function as roots, and in some embodiments may be root hair cell and/or root epidermal cell specific. Root-specific cells are those cells part of the root system of a plant, which may function in a root specific capacity, i.e. anchorage, stability, shelter, nutrient harvesting and storage, water absorption, transport of macromolecules. Underground stems (such as tubers, rhizomes, bulbs, corms, stolons, taro and other plant associated organs which reside underground near the roots) provide similar subterranean functions to botanically-defined root tissues and for the purposes of this invention may be considered as plant tissues that function as roots and comprise root cells.

The carbonic anhydrase may further comprise a transmembrane domain and/or a membrane anchor sequence. The transmembrane domain sequence may be a full length transmembrane domain sequence or a portion thereof. The transmembrane sequence may encode a transmembrane domain which is partially present in the plasma membrane once transported, or it may span the entire plasma membrane. The transmembrane sequence may comprise post-translational elements such as glycoproteins and polysaccharide structures. In some embodiments, the carbonic anhydrase may be attached to the outside of the cell or to the plasma membrane by a transmembrane domain, or a transmembrane helix, or a membrane anchor, or a glycosylphosphatidylinositol (GPI) anchor, or any other biomolecule which causes the carbonic anhydrase to remain attached to the plasma membrane or cell wall following secretion from the cell.

In some embodiments, the transmembrane domain is derived from Pisum sativum BP80. The transmembrane domain sequence encoded in the DNA polynucleotide sequence of the invention may correspond to polynucleotide sequence SEQ ID NO: 5., or a sequence of at least 65% identity with any of said sequences; in some embodiments a sequence of at least 70% identity with any of said sequences; and in some embodiments a sequence of at least 80% identity with any of said sequences or at least 85%, at least 90%, at least 95%, identity with any of said sequences. In some embodiments, the carbonic anhydrase will have any of the % homologies referred to herein and retain the carbonic anhydrase enzymatic activity.

The polynucleotide may correspond to a full-length transmembrane sequence, or a portion thereof. Additionally, the polynucleotide may comprise a sequence that is capable of directing the formation of a glycosylphosphatidylinositol (GPI) anchor, or any other biomolecule which causes the carbonic anhydrase to remain attached to the plasma membrane or cell wall following secretion from the cell.

The term “portion”, “variant”, “homolog” or other representative wording in the art as used herein used in relation to a transmembrane sequence, or a functional fragment thereof, means any transmembrane sequence ortholog of a differing polynucleotide or polypeptide sequence from any species, including plants.

In terms of percentage identity to a reference sequence, such as SEQ ID NO:5, a variant of a transmembrane sequence may have, in increasing order of preference, at least 65%, at least 66%, at least 67%, at least 68%, at least 69%, at least 70%, at least 71%, at least 72%, at least at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% overall sequence identity to that reference sequence.

In some embodiments, the membrane anchor sequence corresponds to an entire glycophosphatidylinositol anchor sequence, or a portion thereof.

Also provided herein is an isolated polynucleotide sequence of the invention which may be fused to another polynucleotide sequence or to a targeting peptide sequence or to a transmembrane domain sequence. The polynucleotide may also be described as being engineered or non-naturally occurring.

Also provided herein is an isolated polypeptide of the invention which may be fused to another polypeptide or to a targeting peptide or to a transmembrane domain. An isolated polynucleotide or polypeptide is a DNA or protein construct which exists on its own separate from the internal environment of the cell. These may comprise DNA or protein associated elements. These can be recombinant. The polypeptide of the invention may transiently comprise a signal peptide, which is subsequently cleaved upon entering the secretory pathway. The polypeptide of the invention may also comprise a signal peptide which represents a first transmembrane domain which acts as a signal peptide, which is not cleaved upon entering the secretory pathway.

Also provided herein are plant cells, cell lines or progeny thereof comprising the polynucleotide sequence or polypeptide sequence disclosed herein. The term “plant” encompasses whole plants, ancestors and progeny of the plants and plant parts, including seeds, fruit, shoots, stems, leaves, roots (including tubers), flowers, and tissues and organs, wherein each comprise the polynucleotide of the invention. The term “plant” also encompasses plant cells, suspension cultures, callus tissue, embryos, meristematic regions, gametophytes, sporophytes, pollen and microspores, wherein each comprise the polynucleotide of the invention.

In some embodiments, the plant is a crop, and in some embodiments a row or cover crop. In some embodiments the plant is selected from a corn, soybean, fava bean, mung bean, adzuki bean, broad bean, pea, cowpea, cotton, canola, camelina, potato, tomato, sugar beet, cassava, yam, sweet potato, alfalfa, wheat, barley, sorghum, oat, sorghum, millet, rye, teff, triticale, miscanthus, sorghum rice, or clover, cress, brassicas (such as Brassica oleracea), vetch and prairie grasses (such as switchgrass).

The plant may be a tree. In some embodiments the tree is poplar, spruce, pine, eucalyptus, oil palm or rubber, particularly those popular in forestry and land management, food production, carbon capture and biofuel or biomass production.

The plant may be an aquatic or a semi-aquatic plant. In some embodiments, such plants may be duckweed, mangroves, nymphaeales, or rice.

The plant, polypeptide, polynucleotide, cell, cell line or progeny of the invention may sequester more carbon into the extracellular environment of the plant in comparison to a plant whose heritable genetic material does not comprise a gene encoding a transportable carbonic anhydrase protein under the control of a promoter active in root cells. Sequestration of carbon occurs via the conversion of CO₂ into bicarbonate and hydrogen ions.

Also provided herein are plant parts, plant tissues, plant organs, plant cells, plant protoplasts, embryos, callus cultures, pollen grains or seeds, derived or obtained from the plant of the invention.

Also provided herein are vectors comprising the polynucleotide sequence of the invention. In some embodiments, the vector is a plasmid. The plasmid may comprise an origin of replication, a T-DNA right border repeat of a Ti or Ri plasmid, a left border repeat of a Ti or Ri plasmid and at least one bacterial selectable marker. The plasmid may also comprise an enhancer, a plant selectable marker, a multicloning site and/or a recombination site.

Compositions for transformation of plant cells comprising the polynucleotide of the invention or the vector comprising the polynucleotide of the invention are also provided herein. The vector may comprise microparticles coated with said polynucleotide or said vector.

Transformation of plants is a routine technique in many species. Any of several transformation methods may be used to introduce a gene of interest into a suitable ancestor cell. According to the various aspects of the invention, the polynucleotide of the invention is introduced into a plant and expressed as a transgene through transformation. The term “introduction” or “transformation” as referred to herein encompasses the transfer of an exogenous polynucleotide into a host cell, irrespective of the method used for transfer. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, floral dipping, biolistics, transformation using viruses or pollen and microinjection. Methods may be selected from electroporation, microinjection, RNA-coated particle bombardment or viral transfection, but are not limited to those methods recited here. The transformation method is usually dependent on the species of choice. The polynucleotide may be transiently or stably introduced into a host cell and may be maintained non-integrated, for example, as a plasmid. Alternatively, it may be integrated into the host genome. The resulting transformed plant cell may then be used to regenerate a transformed plant. To select transformed plants, plant material obtained in the transformation is, as a rule, subjected to selective conditions so that transformed plants can be distinguished from untransformed plants. For example, seeds obtained in the above-described manner can be planted and, after an initial growing period, subjected to a suitable selection and screening.

Also provided herein are bacteria comprising the polynucleotide of the invention, or a vector comprising the polynucleotide of the invention. In some embodiments, the bacterium is E coli. or Agrobacterium sp., and in some embodiments A. tumefaciens. Any suitable cloning system may be used. Genetically modified plants, including crop plants, are in some embodiments produced via Agrobacterium tumefaciens mediated transformation. Such routine methods are also used to introduce gene editing proteins such as CRISPR-Cas nucleases and base editors, which can be used to edit native carbonic anhydrase sequences to be transportable and be increased in expression in root cells.

Also provided herein are plants comprising the polynucleotide of the invention whereby the polynucleotide is stably integrated into the genome thereof; and in some embodiments heritably integrated into the genome thereof.

Methods of producing the plant or seed of the invention are also provided, comprising the steps of:—
(a) providing a vector comprising the polynucleotide of the invention;
(b) activating the promoter operably linked to the polypeptide coding DNA;
(c) optionally wherein the polypeptide is overexpressed in the extracellular environment of the cell in comparison to a plant whose heritable genetic material does not comprise a gene encoding a transportable carbonic anhydrase protein under the control of a promoter active in root cells.

A method for increasing the capacity of a plant to sequester carbon in the soil, the method comprising altering the heritable genetic material of the plant such that a carbonic anhydrase protein is transported from the roots of the plant into the extracellular environment is also provided. The normal capacity of a plant to sequester carbon in the soil is indicated by a plant which has not been genetically modified in the same way as a plant of the present invention. This capacity may reflect the uppermost limit of the rate of conversion of CO₂ into bicarbonate and hydrogen ions in the extracellular environment of plants which have not been genetically modified in the same way as a plant of the present invention. This rate achieved in the modified plants is inherently dependent upon the transport of carbonic anhydrase from the roots of the plant into the extracellular environment. The altering of heritable genetic material may comprise inserting at least one polynucleotide into the heritable genetic material of a plant cell using methods of transgenic manipulation is well known in the art.

The altering of heritable genetic material may also comprises genetically editing the heritable genetic material of a plant cell, using methods of genetic modification well known in the art, such as CRISPR, TALENs and meganucleases. Base editing may also be used, whereby a single nucleotide can be manipulated in a targeted gene. Prime editing may also be used, which is a search-and-replace genome editing method used to insert precise, small indels including single or multiple base substitutions, transitions and transversions without requiring double stranded breaks and donor repair templates. Any regulatory sequence may also be manipulated using these techniques to result in increased expression of the carbonic anhydrase, for example, by introducing random indels into a native regulatory sequence using a CRISPR-Cas system.

Any polynucleotides disclosed herein may further include a polynucleotide sequence encoding a CRISPR-Cas protein, optionally a guide RNA (gRNA), wherein the gRNA directs the CRISPR-Cas protein to the locus of at least one copy of an endogenous carbonic anhydrase gene thereof in the plant cell genome, whereby a root-specific promoter is inserted so as to cause expression of the copy or copies of the carbonic anhydrase in at least some root cells of the plant.

A native carbonic anhydrase may also be manipulated using gene editing tools to introduce a signal peptide to a native carbonic anhydrase gene using targeted nucleases and homology directed repair or non-homologous end joining repair mechanisms and a template sequence containing the signalling peptide. Such gene knock in techniques are well known in the art.

Also provided herein are processed plant products obtained from the plant or the plant part, plant tissue, plant organ, plant cell, plant protoplast, embryo, callus culture, pollen grain or seed disclosed herein. The processed plant product may comprise a detectable nucleic acid sequence of (i) a transportable carbonic anhydrase downstream of a gene expression regulatory element active in at least some of the root cells of a plant, or (ii) a polynucleotide or at least a portion of a polynucleotide of the invention which is present in the product. Processing may involve chemical or physical manipulation of the product in order to form a product with desirable properties suited to a particular function, i.e. pelleting.

### EXAMPLES

### Example 1

To demonstrate that plant roots can be engineered to produce active carbonic anhydrase protein a soybean (Glycine max) carbonic anhydrase gene was selected for overexpression (accession number Glyma.19G135900 [SEQ ID NO: 1 and SEQ ID NO: 8]). This carbonic anhydrase has an annotated ‘Eukaryotic-type carbonic anhydrase’ domain (Pfam #PF00194.24) in its protein sequence.

The expression of the carbonic anhydrase gene (SEQ ID NO: 1 and SEQ ID NO: 8) was placed under the control of a 35S promoter and cloned into the pCAMBIA-1300 plant expression vector. This vector included a herbicide resistance gene (kanamycin) and the expression cassette was flanked by agrobacterium LB and RB t-DNA sequences. The vector was transformed into Agrobacterium rhizogenes strain K599 using electroporation and this was used to transform soybean roots using a hairy root transformation method according to the protocols in (Chen et al., 2018).

Following transformation positively transformed root tissue was identified visually by the expression of a visual reporter. Root tissue was isolated from transformed roots and equivalent non-transformed root tissue was sampled as a control. Root tissue was homogenised in in 700 μl of extraction buffer in a chilled pestle and mortar. Homogenised root was transferred to 2 ml tube and centrifuged for 15 mins at 5000 rpm at 4 C. All assays were performed on the supernatant following this spin step.

A carbonic anhydrase assay was carried out to measure carbonic anhydrase activity. This assay measures the rate of change in pH of CO₂-saturated water following the addition of root homogenate from transformed and untransformed (also known as wild type control) root tissue. The pH change results from the release of protons (H⁺) by carbonic anhydrase. Change in pH is directly proportional to the amount of bicarbonate that has been produced by carbonic anhydrase. Such assays are known to those familiar with the art.

For each carbonic anhydrase assay 100 μl of supernatant from the homogenised root samples was used. For a no-root tissue control, 100 μl of extraction buffer without root tissue was used. Samples were added to 2 ml tube containing 1100 μl of a 20 mM Tris-HCl solution at pH 8.3 containing 0.003% bromothymol blue. Following this, 800 μl of ice chilled CO₂ saturated water was added. The time taken to change colour from blue (pH 8.3) to yellow (pH 5.3) was recorded. All reactions were performed on ice.

The Wilbur-Anderson Units (WA) equation was used to calculate activity:

$$WA units = ( t 0 - ts ) / ts$$

where t0 is the time taken for the colour change to occur in the no-root control reaction and ts is the time taken for the colour change to occur in samples containing supernatant from the homogenised root.

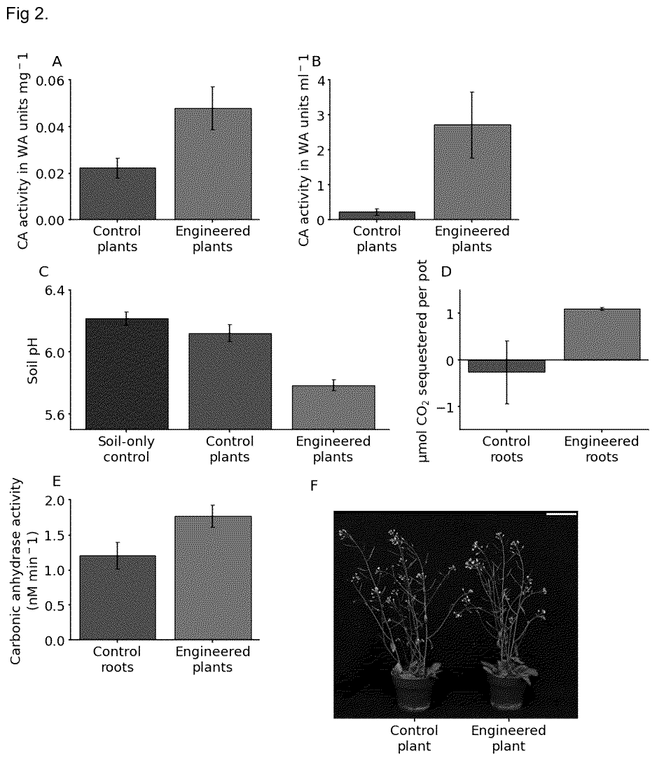

Carbonic anhydrase activity was significantly increased at ~2× higher in the transgenic root samples compared to the wild type root samples (P=0.022669, one-tailed Student's t-test, unequal variance) (FIG. 2A). Thus, soybean root cells can be engineered to have increased carbonic anhydrase activity through the overexpression of a gene encoding a carbonic anhydrase protein, and this protein is catalytically active when released from root cells into an extracellular environment.

### Example 2

In order for carbonic anhydrase to facilitate conversion of CO₂ to HCO₃ in the extracellular environment of the plant they need to be secreted/transported from the cells of the plant into the extracellular environment. There are several mechanisms by which cells target proteins for secretion/transport from the cell into the extracellular environment. The two main ways in which this is achieved are the conventional secretory pathway and the unconventional secretory pathway. The conventional secretory pathway achieves protein secretion/transport through the use of N-terminal signal peptides.

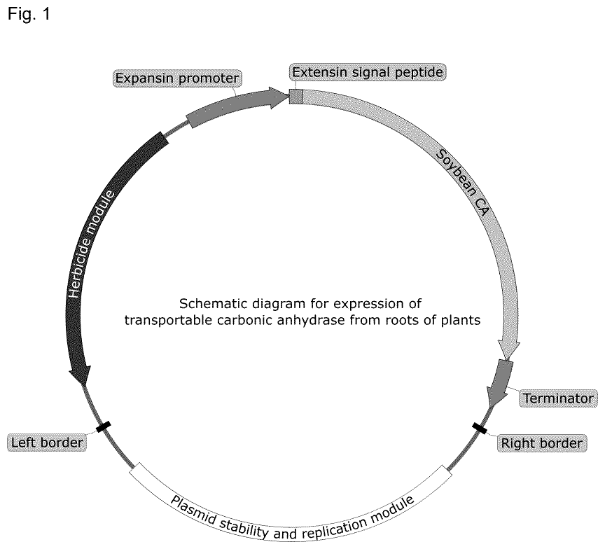

To demonstrate that carbonic anhydrase proteins can be engineered to be secreted/transported from plant cells we translationally fused an experimentally characterised signal peptide from the carrot (Daucus carota) extensin gene (G. Li et al., 2009) [SEQ ID NO: 3] with the soybean carbonic anhydrase provided in Example 1 [SEQ ID NO: 1 and SEQ ID NO: 8]. This fusion protein was placed under the control of a root specific promoter from the soybean expansin gene (Glyma.17G133100) [SEQ ID NO:6], and cloned into a plant transformation vector that also included a herbicide resistance gene (spectinomycin) and was flanked by agrobacterium LB and RB t-DNA sequences (FIG. 1). The vector was transformed into Agrobacterium tumefasciens (LBA4404 strain) by electroporation, positive colonies were selected and cultured using standard techniques. To demonstrate that this technology would have broad utility in all plant species, this vector was transformed into Arabidopsis thaliana using floral dip method (Clough & Bent, 1998) and stable transgenic lines were generated.

It should be noted that although the soybean expansin gene promoter was used in this example, other root promoters are known in the art (for example (Y. Li et al., 2019; Marquès-Bueno et al., 2016). It would be expected by those of ordinary skill in the art that any root promoter selected from any species would be expected to function equivalently to drive expression in roots. It should also be noted that the technology of the invention would be expected to work if the carbonic anhydrase protein was expressed throughout the plant, or in other plant tissues, so long as the expression domain encompassed expression in roots.

An experiment was performed to demonstrate that plants expressing secretable/transportable carbonic anhydrase in root cells resulted in carbonic anhydrase activity in the extracellular environment of the root. Here, transformed plants and wild type control plants were grown for six-weeks in hydroponic conditions. At six weeks, individual plants were transferred to 15 ml of fresh hydroponic media and allowed to grow for two days. Following this two-day period, the hydroponic solution was sampled and used for carbonic anhydrase activity assays to measure the amount of carbonic anhydrase activity that had been secreted/transported from the roots into the hydroponic medium.

Here, 100 μl of hydroponic solution was added to a 2 ml snap lid microcentrifuge tube containing 1050 μl of a 20 mM Tris-HCl solution (pH 8.3) with 0.003% bromothymol blue and 100 μl extraction buffer (200 mM Tris HCl pH8.3, 1 mM EDTA, 20 mM MgCl₂, 50 mM NaCl, 100 mM Na₂SO₄). Subsequently, 750 μl of ice chilled CO₂ saturated water (generated by soda stream) was added to start the reaction. The time taken for a colour change from blue (pH 8.3) to yellow (pH 5.3) was recorded and used to calculate enzyme activity as in Example 2. The reaction was performed on ice.

The carbonic anhydrase activity was significantly increased at 11× higher in the hydroponic medium samples from transgenic plants than from hydroponic medium samples from wild-type control plants (P=0.029404, one-tailed Student's t-test, unequal variance) (FIG. 2B). Thus, plants transformed with the invention successfully expressed and secreted active carbonic anhydrase protein into the extracellular environment of the root, which in turn catalysed conversion of CO₂ to bicarbonate.

### Example 3

To demonstrate that this technology also works to sequester CO₂ in the soil a separate experiment was performed on soil grown plants. Given that carbonic anhydrase catalyses the conversion of CO₂ and water into bicarbonate and a proton, the sequestration of atmospheric CO₂ in the soil is directly proportional to the pH change of the soil. Here, transgenic plants and wild type plants (described in Example 2) were grown in 6.5 cm pots containing 110 g Westland John Innes No. 3 Mature Plant Compost for six weeks. Soil control pots not containing any plants were also constructed. For each pot, the pH of the soil 2 cm below the surface was measured in three random locations using a HANNA™ soil pH tester (HI-981030). As shown in FIG. 2C, the soil pH in the pots containing transgenic plants of the invention was significantly lower than either pots containing no plants (‘soil control’) (P=0.000092, one-tailed Student's t-test, unequal variance). or pots containing wild-type plants (P=0.000449, one-tailed Student's t-test, unequal variance). Thus, pots containing plants of the invention sequestered more CO₂ in the soil than pots containing control plants or pots containing no plants.

Plants were also imaged as they grew. There were no obvious differences in the phenotype between transgenic and wild type plants across plant development, growth rate, flowering, or seed set (FIG. 2F). Thus, secreting carbonic anhydrase from roots, and sequestering CO₂ in the soil as bicarbonate, is not deleterious to plant growth.

### Example 4

A CO₂ soil flux experiment was conducted to further demonstrate that transgenic plants of the invention increased the rate at which CO₂ diffuses from the air into the soil and is converted into bicarbonate.

Here, transgenic plants and wild type plants (described in Example 2) were grown in 6.5 cm pots containing 110 g Westland John Innes No. 3 Mature Plant Compost for 6 weeks. After 6 weeks all above ground biomass (i.e. leaves, stems etc) was removed and the pot containing all of the below ground biomass in soil was placed in a LI-COR 6800 small plant chamber in complete darkness (achieved by covering chamber in tin-foil). The CO₂ that was absorbed or emitted by the soil in the pot was then measured using the LI-COR 6800. To do this, the chamber was equilibrated in CO₂ free air for 1 hour. After 1 hour, the CO₂ level was raised to 1000 ppm (~2.5× atmospheric CO₂ concentration). Following 1 minute of acclimation, the soil CO₂ sequestration rate was measured every second for a 30-minute recording window. Net carbon sequestration was calculated by summing the carbon sequestration over the recording window. As shown in FIG. 2D, this revealed that pots containing control plant roots did not sequester CO₂ from the air stream, and instead produced CO₂ through soil respiration. In contrast, pots containing roots of transgenic plants of the invention depleted CO₂ from the air stream. Thus, plants of the invention facilitated CO₂ sequestration into the soil. Moreover, the magnitude of this sequestration is substantially larger than the CO₂ released through soil respiration.

### Example 5

To demonstrate that other carbonic anhydrase genes from other species can be used in different embodiments of this invention we expressed a carbonic anhydrase from the diatom Conticribra weissflogii (also known as Thalassiosira weissflogii) [SEQ ID NO: 2]. As above this carbonic anhydrase was translationally fused to the experimentally characterised signal peptide from the carrot (Daucus carota) extensin gene (G. Li et al., 2009) [SEQ ID NO: 3]. This fusion protein was placed under the control of a root specific promoter from the soybean expansin gene (Glyma.17G133100) [SEQ ID NO:6], and cloned into a plant transformation vector that also included a herbicide resistance gene (spectinomycin) and was flanked by agrobacterium LB and RB t-DNA sequences. The vector was transformed into Agrobacterium tumefasciens (LBA4404 strain) by electroporation, positive colonies were selected and cultured using standard techniques. This vector was transformed into Arabidopsis thaliana and stable transgenic lines were generated.

Seeds of transgenic plants expressing the secretable/transportable carbonic anhydrase from Conticribra weissflogii (SEQ ID NO 2 and 9) and seeds of wild type plants were germinated on ½ Murashige and Skoog+1% sucrose+2.5 mM MES agar plates. After 7 days seedlings were transferred to 96 well plates with only the roots entering each well. Each well in the 96 well plate contained 350 μl of ½ Murashige and Skoog+1% sucrose solution and 30 mg L⁻¹ bromocresol purple. After four hours the seedlings were removed and the absorbance at 429 nm was measured using a plate reader. Carbonic anhydrase activity was estimated from the rate of change in pH over the four hour time period. The carbonic anhydrase activity was significantly higher in the root environment of the transgenic plants than in the root environment of the wild-type control plants (P=0.017435, one-tailed Student's t-test, unequal variance) (FIG. 2E). Thus, plants transformed with the secretable/transportable carbonic anhydrase from Conticribra weissflogi successfully expressed and secreted active carbonic anhydrase protein into the extracellular environment of the root, which in turn catalysed conversion of CO₂ to bicarbonate. This demonstrates that carbonic anhydrase proteins from any species and any carbonic anhydrase subtype would be by expected to work equivalently.

### Example 6

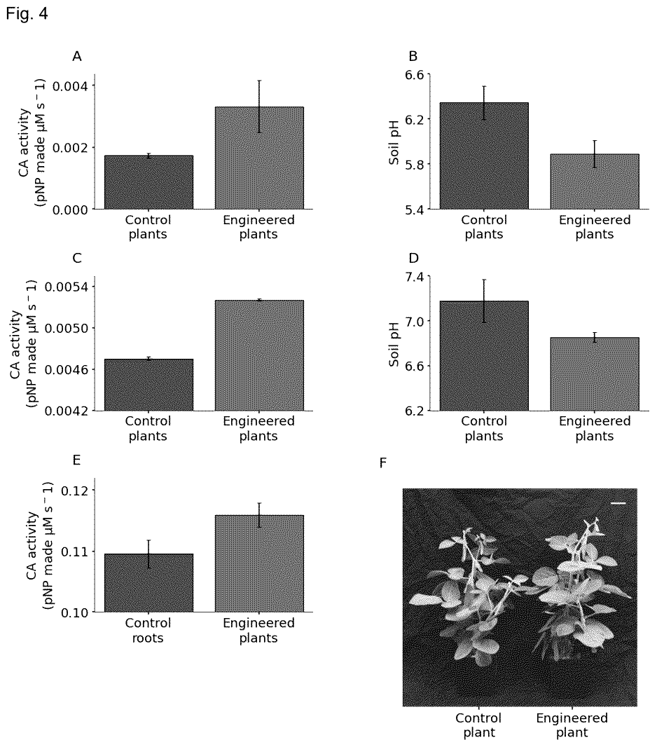

To demonstrate that soybean plants can be engineered such that their roots secrete active carbonic anhydrase proteins into the extracellular environment we translationally fused an experimentally characterised signal peptide from the carrot (Daucus carota) extensin gene provided in Example 2 [SEQ ID NO: 3] with the soybean carbonic anhydrase provided in Example 1 [SEQ ID NO: 1 and SEQ ID NO: 8]. This fusion protein was placed under the control of a root specific promoter from the soybean expansin gene (Glyma.17G133100) [SEQ ID NO: 6], and cloned into a plant transformation vector that also included a herbicide resistance gene (spectinomycin) and was flanked by agrobacterium LB and RB t-DNA sequences (FIG. 1). The vector was transformed into Agrobacterium tumefasciens cultured using standard techniques. This vector was transformed into Glycine max and stable engineered lines were generated. Engineered and azygous control plants were grown for 3-weeks on Levington advance F2S Seed & Modular & Sand Compost before washing roots and transplanted to 4.5 L hydroponic boxes. Plants were grown on hydroponic media for two weeks as previously described by Hata & Futamura (2020). After two weeks the hydroponic media was replenished with fresh media and plants were grown for five days depending on the rate of plants consuming hydroponic media. Samples of hydroponic media from hydroponic boxes containing either control or engineered plants were filtered through a Falcon 40 μm cell strainer to remove plant debris. Proteins present in the filtered samples were then isolated and concentrated using Pierce 10 kD MWCO protein concentrating column. The total protein concentration of each isolated protein sample was calculated using a Bradford Protein assay and total protein concentration adjusted to be equal across all samples. To measure Carbonic anhydrase activity an adapted version of the protocol described in Ozdemir ((2009) was used. Here, 160 μl of concentrated hydroponic media samples were added to 120 μl of 50 mM Tris-HCl pH 7.5 in a well of a 96-well microplate. Following this, 30 μl of 10 mM p-Nitrophenyl acetate dissolved in acetonitrile was added to each well to start the colorimetric assay. A spectrophotometer plate reader was used to measure absorbance in each well at 400 nm over a 16-hour period at 25 C. Carbonic anhydrase activity was reported as μM of p-Nitrophenol synthesised per second. Mean carbonic anhydrase activity was calculated from two independent azygous lines and three independent engineered lines (FIG. 4A). Protein samples isolated from hydroponic medium that contained azygous plants exhibited a baseline p-nitrophenyl hydrolysis activity. Protein samples isolated from hydroponic medium that contained engineered plants exhibited a significantly higher p-nitrophenyl hydrolysis activity.

This is consistent with the presence of secreted carbonic anhydrase in the hydroponic medium that contained engineered plants.

Plants were also imaged as they grew on soil. There were no obvious differences in the phenotype between engineered soybean plants and control plants across plant development, growth rate, flowering, or seed set (FIG. 4F). Thus, secreting carbonic anhydrase from roots, and sequestering CO₂ in the soil as bicarbonate, was not deleterious to plant growth.

### Example 7

To demonstrate that soybean plants that have been engineered to secrete active carbonic anhydrase proteins into the extracellular environment enable sequestration of CO₂ in the soil the following experiment was performed. Engineered plants expressing a secretable carbonic anhydrase in root hair cells (as described in Example 6) and wild-type control plants were grown in 13 cm pots containing 1:1:2:2 Levington M3 compost, Melcourt topsoil, Melcourt sharp sand and perlite mixed with 5 ml Miracle-Gro All Purpose Continuous Release Plant Food. When plants were 17-weeks old a 2.1 cm diameter soil sampler probe was used to collect a soil core at a random location near the root zone from each pot and transferred to falcon tube. An equal volume to soil weight of ddH2O was added and mixed for 30 minutes and allowed to stand for 1-hour. Using a METLER pH probe the pH of the soil slurry was measured. The pH of the soil from the pots containing engineered plants of the invention was significantly lower (P=0.00798, one-tailed Student's t-test, unequal variance) than pots containing wild-type plants (FIG. 4B). This reduction in soil pH is consistent with the presence of active carbonic anhydrase protein, and thus an increased concentration of bicarbonate, in the soil of pots containing engineered plants of the invention. Thus, soil in which plants of the invention were grown sequestered more CO₂ than soil in which control plants were grown.

### Example 8

To demonstrate that carbonic anhydrase genes from any species can be used in engineered plants of the invention the soybean carbonic anhydrase gene used in Example 1 and Example 2 above was exchanged for a carbonic anhydrase gene from a marine alga [SEQ ID NO: 2]. All other components of the vector for plant transformation were kept the same. To demonstrate that the carbonic anhydrase protein that is expressed is secreted from soybean roots and is active engineered plants of the invention and azygous control plants were grown for 3-weeks on Levington advance F2S Seed & Modular & Sand Compost before washing roots and transplanting to 4.5 L hydroponic boxes. Plants were supported on hydroponic media as previously described by (Hata & Futamura 2020). Plants were grown hydroponically for 1-week. Hydroponic media was then replenished with fresh media and plants were grown for a further 7-days. Samples of media from hydroponic boxes containing only control or engineered plants was isolated and filtered through a Falcon 40 μm cell strainer to remove plant debris. The proteins present in the filtered samples were then isolated and concentrated using Pierce 10 kDMWCO protein concentrating column. The total protein concentration of each sample was calculated using a Bradford Protein assay and enzyme activity was normalised to total protein content. To measure Carbonic anhydrase activity an adapted version of protocol described in Ozdemir (2009). was used. Here, 160 μl of concentrated hydroponic media samples were added to 120 μl of 50 mM Tris-HCl pH 7.5 in a well of a 96-well microplate. Following this, 30 μl of 10 mM p-Nitrophenyl acetate dissolved in acetonitrile was added to each well to start the colorimetric assay. A spectrophotometer plate reader was used to measure absorbance in each well at 400 nm over a 11-hour period at 25 C.

As shown in FIG. 4C, protein samples isolated from hydroponic medium that in which azygous plants were grown exhibited a baseline p-nitrophenyl hydrolysis activity. Protein samples isolated from hydroponic medium that in which engineered plants of the invention were grown exhibited a significantly higher p-nitrophenyl hydrolysis activity. This is consistent with the presence of secreted carbonic anhydrase in the hydroponic medium that contained engineered plants.

### Example 9

To demonstrate that soybean plants that have been engineered to express active carbonic anhydrase proteins on the surface of their roots to enable sequestration of CO₂ in the soil, the following experiment was performed. As above engineered soybean plants where soybean carbonic anhydrase [SEQ ID NO: 3] was translationally fused with experimentally characterised signal peptide and transmembrane domain from pea (Pisum sativum) [SEQ ID NO: 4 and SEQ ID NO:5]. This fusion protein was placed under the control of a root specific promoter from the soybean expansin gene (Glyma.17G133100) [SEQ ID NO:6], and cloned into a plant transformation vector that also included a herbicide resistance gene (spectinomycin) and was flanked by agrobacterium LB and RB t-DNA sequences (FIG. 1).

The vector was transformed into Agrobacterium tumefasciens cultured using standard techniques. This vector was transformed into Glycine max and stable engineered lines were generate. Engineered and azygous control plants were grown in 13 cm pots containing John Innes Cereal Mix. When plants were 13-weeks old a 2.1 cm diameter soil sampler probe was used to collect a soil core sample at a random location near the root zone from each pot and transferred to falcon tube. An equal volume to soil weight of ddH2O was added and mixed for 30 minutes and allowed to stand for 1-hour. The pH of the soil slurry was measured using a METLER pH probe. As shown in FIG. 4D, the pH of the soil in the pots in which engineered plants of the invention were grown was significantly lower than the pH of the soil in the pots containing wild-type plants. This reduction in soil pH is consistent with the presence of active carbonic anhydrase protein, and thus an increased concentration of bicarbonate, in the soil of pots containing engineered plants of the invention. Thus, pots containing engineered plants of the invention sequestered more CO₂ in the soil than pots containing control plants or pots containing no plants.

### Example 10

To demonstrate that other carbonic anhydrase genes from other species can be engineered and localised extracellularly on root hairs of soybean roots engineered plants used in FIG. 4C and their respective azygous control plants were grown for 3-weeks on Levington advance F2S Seed & Modular & Sand Compost and transplanted to 4.5 L hydroponic boxes. Plants were supported on hydroponic media as previously mentioned by Hata & Futamura (2020). Plants were grown hydroponically for 2-weeks with media refreshed weekly. To measure carbonic anhydrase activity on root surface an adapted carbonic anhydrase p-nitrophenyl hydrolysis activity protocol taken from Ozdemir (2009) was used. 500 mg fresh weight of soybean roots were removed were briefly washed with sterile water to remove excess hydroponic media and then placed in 30 ml beaker containing 18 ml 50 mM Tris-HCl pH 7.5. 2 ml 10 mM p-Nitrophenyl acetate dissolved in acetonitrile was used to start the reaction. Absorbance of 300 μl samples at 400 nm was recorded at 1-minute and 33 minutes using spectrophotometer plate reader. Carbonic anhydrase activity was reported as μM of p-Nitrophenol synthesised per second.

Engineered plant roots demonstrated significantly higher (P=0.030458, one-tailed Student's t-test, unequal variance) p-nitrophenyl hydrolysis activity compared to control plant roots (FIG. 4E). This is consistent with the detection of carbonic anhydrase activity extracellularly from roots of engineered plants.

### Example 11

To demonstrate that carbonic anhydrase enzymes can be engineered to be localised extracellularly on the plasma-membrane of the cell, a reporter-gene fusion was made and tested in protoplasts. Here, Conticribra weissflogii carbonic anhydrase [SEQ ID NO: 2] was translationally fused with the coding sequence for a fluorescent protein to produce a gene-reporter fusion. This gene-reporter fusion was then translationally fused at the N-terminus and C-terminus with a signal peptide and transmembrane domain, respectively, from the pea (Pisum sativum) gene PsBP80 [SEQ ID NO: 4 and SEQ ID NO:5]. This final fusion protein was placed under the control of a cassava vein mosaic virus promoter (Verdaguer et al. (1996)) and cloned into a plant transformation vector. To provide a cytoplasmic localised control, the Conticribra weissflogii carbonic anhydrase translationally fused with the coding sequence for a fluorescent protein, without the PsPB80 signal peptide and transmembrane domain, was also placed under the control of a cassava vein mosaic virus promoter (Verdaguer et al. (1996)) and cloned into a plant transformation vector.

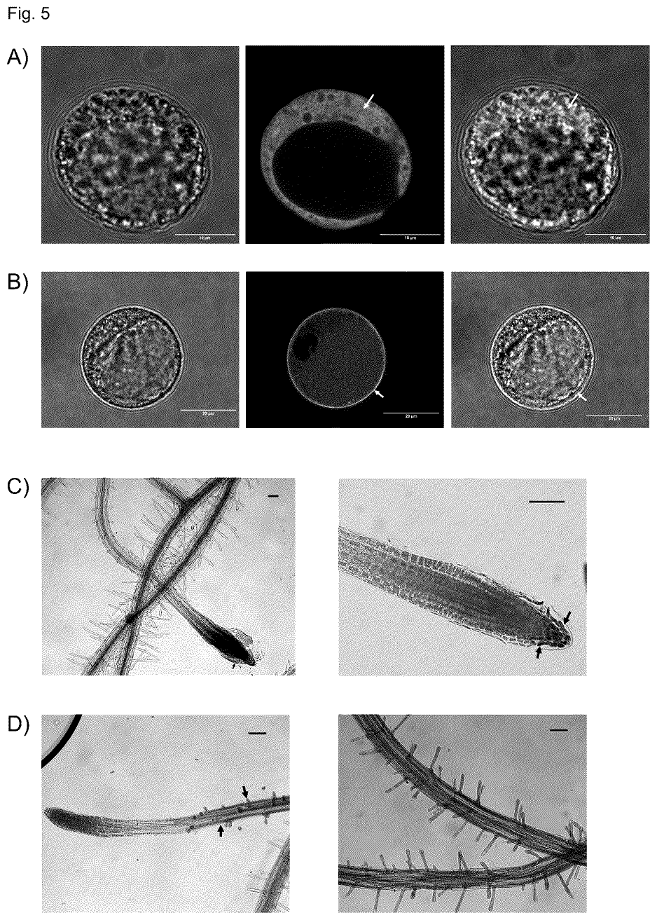

Soybean protoplasts were transfected with these two expression vectors following the protocol by Sultana et al. (2019). After 2-days, the transfected protoplasts were imaged using a Leica TCS SP5 confocal microscope. Protoplasts expressing Conticribra weissflogii carbonic anhydrase fused to the coding sequence for a fluorescent protein, without the PsPB80 signal peptide and transmembrane domain, showed a clear cytosolic localisation (FIG. 5A). In contrast, protoplasts expressing Conticribra weissflogii carbonic anhydrase fused to the coding sequence for a fluorescent protein with the PsBP80 signal peptide at the N-terminus and the PsBP80 transmembrane domain at the C-terminus localised to the plasma membrane (FIG. 5B).

### Example 12

To demonstrate that promoters used for generation of engineered plants herein drive expression in specific root tissues, GUS reporter lines were generated. Here, pGmEXPA7 (SEQ ID NO:6) and pGmPIN2b (SEQ ID NO:7) were each used independently to drive expression of GUS (Cat1+) (beta-glucuronidase gene with castor bean catalase first intron Cat1). These promoter-GUS fusions were separately cloned into a plant transformation vector that included a herbicide resistance gene (Kanamycin) and was flanked by agrobacterium LB and RB t-DNA sequences. The resulting vectors were transformed into Agrobacterium tumefasciens (LBA4404 strain) by electroporation and positive colonies were selected and cultured using standard techniques. These agrobacterium strains were used to transform Arabidopsis thaliana via the floral dip method (Clough & Bent, 1998). Engineered plants were germinated on ½ Murashige and Skoog+1% sucrose+2.5 mM MES agar plates. After 10 days, seedlings were transferred to 6-well plates and GUS histochemical staining was performed. 5 ml of GUS staining buffer (0.5 mM K₃Fe(CN) 6, 0.5 mM K₄Fe(CN)₆ 3H₂O, 50 mM Sodium Phosphate buffer, 10 mM Na₂EDTA H₂O, 1 mM X-Gluc and 0.1% Triton-X (v:v)) was added to seedlings and incubated at 37 C for 3-hours. Seedlings were washed and cleared with 70% ethanol overnight. Stained and chlorophyll cleared seedlings roots were imaged using an Optika B-383FL microscope combined with OneView camera and imaging software.

GUS expression driven by the pGmPIN2b promoter [SEQ ID NO:6) was localised to the epidermis of root tip and some expression extending up the primary root (FIG. 5C). GUS expression driven by the pGmEXPA7 promoter was localised to the root hair cells (FIG. 5D).

### Example 13

To demonstrate that engineered plants of the invention enable increased accumulation of soil inorganic carbon the following experiment was performed. Engineered plants described in Example 6 that express a secreted soybean carbonic anhydrase from root hair cells [SEQ ID NO: 6] and azygous control plants were grown in 13 cm pots containing 1:1:2:2 Levington M3 compost, Melcourt topsoil, Melcourt sharp sand and perlite soil mixed with 5 ml Miracle-Gro All Purpose Continuous Release Plant Food until the end-of-life cycle. When seeds were harvested, and soil was dry, the dried biomass and roots in the soil were removed and the remaining soil was sieved through 2 mm sieve. The soil samples were sent to Eurofins AgroTesting UK for soil Carbon Check to analyse soil inorganic (SIC) and organic carbon (SOC) content.

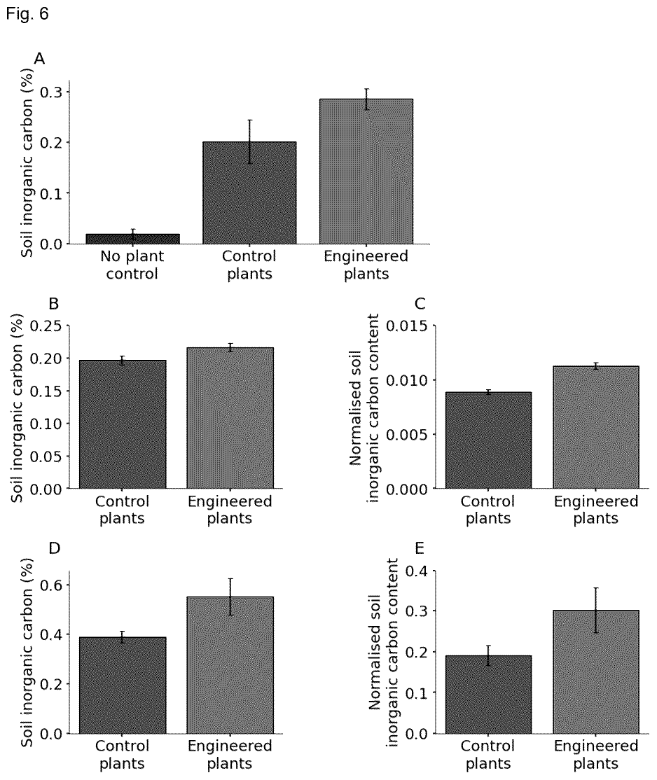

Mean inorganic carbon content of soil from pots in which engineered plants were grown was higher than soil from pots in which azygous control plants or no plants were grown (FIG. 6A).

### Example 14

To demonstrate that exogenous soil treatments can be used in combination with engineered plants of the invention to enhance CO₂ sequestration in the soil the following experiment was performed. Here, engineered Arabidopsis thaliana plants described in Example 2 expressing a secretable carbonic anhydrase in root hair cells and wild-type control plants were grown in 6.5 cm pots containing 160 g of Levington advance F2S Seed & Modular & Sand Compost mixed with 4.16 g L−1 Ca(OH)₂. The pH of the soil was adjusted to pH 8 to facilitate enhanced conversion of HCO₃— to CO₃— which can then combine with Ca from Ca(OH)₂ to form CaCO₃.

Plants were grown for 10-weeks. At 10 weeks, all above soil biomass was removed and the soil was sent to Eurofins AgroTesting UK for soil Carbon Check to analyse soil inorganic (SIC) and organic carbon (SOC) content. Soil in which engineered plants of the invention were grown exhibited increased soil inorganic carbon (FIG. 6B) and significantly higher soil inorganic carbon normalised against soil organic carbon (P=0.002078, one-tailed Student's t-test, unequal variance) (FIG. 6C). This is consistent with the presence of carbonic anhydrase activity, and thus enhanced conversion of CO₂ to bicarbonate, in the soil in which engineered plants of the invention were grown. Thus, soil treatments can be used in conjunction with engineered plants of the invention to enhance CO₂ sequestration. It would be expected by those of ordinary skill in the art that any soil treatment rich in positively charge cations such as magnesium oxide or cations derived from crushed rock soil treatments or fertilizer treatments or deploying the technology on soils that are naturally richer in cations would be expected to function equivalently when used with our invention to increase inorganic soil carbon. our invention to increase inorganic soil carbon.

### Example 15

To demonstrate that engineered plants of the invention enable increased accumulation of soil inorganic carbon the following experiment was performed. Engineered soybean plants described in Example 8 expressing soybean carbonic anhydrase that is anchored to the plasma membrane of root hair cells and their respective azygous control plants were grown in 13 cm pots containing 1:1:2:2 Levington M3 compost, Melcourt topsoil, Melcourt sharp sand and perlite soil mixed with 5 ml Miracle-Gro All Purpose Continuous Release Plant Food until the end-of-life cycle. When seeds were harvested, and soil was dry, the dried biomass and roots in the soil were removed and the remaining soil was sieved through 2 mm sieve. The soil samples were sent to Eurofins AgroTesting UK for soil Carbon Check to analyse soil inorganic (SIC) and organic carbon (SOC) content.

Soil in which engineered plants of the invention were grown exhibited significantly increased soil inorganic carbon (P=0.032395, one-tailed Student's t-test, unequal variance) (FIG. 6D) and significantly higher soil inorganic carbon normalised against soil organic carbon (P=0.048304, one-tailed Student's t-test, unequal variance) (FIG. 6E) than soil in which control plants were grown.

This is consistent with the presence of increased carbonic anhydrase activity, and thus enhanced conversion of CO₂ to bicarbonate to generate inorganic carbon in the soil in which plants of the invention were grown. Thus, demonstrating engineered plants of the invention to enhance carbon sequestration in the soil.

### REFERENCES

Boone, C. D., Gill, S., Habibzadegan, A., & McKenna, R. (2013). Carbonic anhydrase: An efficient enzyme with possible global implications. In International Journal of Chemical Engineering. https://doi.org/10.1155/2013/813931
Chen, L., Cai, Y., Liu, X., Guo, C., Sun, S., Wu, C., Jiang, B., Han, T., & Hou, W. (2018). Soybean hairy roots produced in vitro by Agrobacterium rhizogenes-mediated transformation. The Crop Journal, 6 (2), 162-171. https://doi.org/10.1016/j.cj.2017.08.006
Clough S., Bent A. Floral dip: a simplified method for Agrobacterium-mediated transformation of Arabidopsis thaliana. Plant J. 1998, 16 (6), 735-743. doi: 10.1046/j.1365-313x.1998.00343.x. PMID: 10069079.
DiMario, R. J., Clayton, H., Mukherjee, A., Ludwig, M., & Moroney, J. v. (2017). Plant Carbonic Anhydrases: Structures, Locations, Evolution, and Physiological Roles. Molecular Plant, 10 (1), 30-46. https://doi.org/10.1016/j.molp.2016.09.001
Hayashi, T., Shimoda, Y., Sato, S., Tabata, S., Imaizumi-Anraku, H., & Hayashi, M. (2014). Rhizobial infection does not require cortical expression of upstream common symbiosis genes responsible for the induction of Ca²⁺ spiking. Plant Journal, 77 (1), 146-159. https://doi.org/10.1111/tpj.12374
Hata, N., & Futamura, H. (2020). PRODUCTION OF SOYBEAN PLANTS FOR HYDROPONIC CULTIVATION FROM SEEDLING CUTTINGS IN A MEDIUM CONTAINING RHIZOBIUM INOCULUM DEPENDING ON VARIOUS CONCENTRATIONS OF NUTRIENT SOLUTION AND DIFFERENT NITROGEN SOURCES. Journal of Horticultural Research, 28 (2), 71-82.
Hirakawa, Y., Senda, M., Fukuda, K., Yu, H. Y., Ishida, M., Taira, M., Kinbara, K., & Senda, T. (2021). Characterization of a novel type of carbonic anhydrase that acts without metal cofactors. BMC Biology, 19 (1). https://doi.org/10.1186/s12915-021-01039-8
Horowitz, C. A. (2016). Paris Agreement. International Legal Materials, 55 (4), 740-755. https://doi.org/10.1017/S0020782900004253
Li, G., Yang, S., Li, M., Qiao, Y., & Wang, J. (2009). Functional analysis of an Aspergillus ficuum phytase gene in Saccharomyces cerevisiae and its root-specific, secretory expression in transgenic soybean plants. Biotechnology Letters, 31 (8), 1297-1303. https://doi.org/10.1007/s10529-009-9992-6
Li, Y., Liu, X., Chen, R., Tian, J., Fan, Y., & Zhou, X. (2019). Genome-scale mining of root-preferential genes from maize and characterization of their promoter activity. BMC Plant Biology, 19 (1). https://doi.org/10.1186/s12870-019-2198-8
Marqués-Bueno, M. M., Morao, A. K., Cayrel, A., Platre, M. P., Barberon, M., Caillieux, E., Colot, V., Jaillais, Y., Roudier, F., & Vert, G. (2016). A versatile Multisite Gateway-compatible promoter and transgenic line collection for cell type-specific functional genomics in Arabidopsis. Plant Journal, 85 (2), 320-333. https://doi.org/10.1111/tpj.13099
Masson-Delmotte, V., P. Z. H.-O. P. D. R. J. S. P. R. S. A. P. W. M.-O. C. P. R. P. S. C. J. B. R. M. Y. C. X. Z. M. I. G. E. L. T. M. M. T. and T. W. (2018). IPCC, 2018: Summary for Policymakers. In Global Warming of 1.5° C. An IPCC special report. (pp. 3-24). Cambridge University Press. https://doi.org/10.1017/9781009157940.001
Matt, A.-S., Chang, W. W., & Hu, M. Y. (2022). Extracellular carbonic anhydrase activity promotes a carbon concentration mechanism in metazoan calcifying cells. Proceedings of the National Academy of Sciences, 119 (40). https://doi.org/10.1073/pnas.2203904119
Ozdemir, E. (2009 August 7). Biomimetic CO₂ Sequestration: 1. Immobilization of Carbonic Anhydrase within Polyurethane Foam. Energy Fuels, 23, 5725-5730. doi: 10.1021/ef9005725
Steger, F., Reich, J., Fuchs, W., Rittmann, S. K. M. R., Gübitz, G. M., Ribitsch, D., & Bochmann, G. (2022). Comparison of Carbonic Anhydrases for CO2 Sequestration. International Journal of Molecular Sciences, 23 (2). https://doi.org/10.3390/ijms23020957
Strefler, J., Amann, T., Bauer, N., Kriegler, E., & Hartmann, J. (2018). Potential and costs of carbon dioxide removal by enhanced weathering of rocks. Environmental Research Letters, 13 (3). https://doi.org/10.1088/1748-9326/aaa9c4
Sultana, S. M., Frazier, T. P., Millwood, R. J., Lenaghan, S. C., & Stewart Jr, N. C. (2019). Development and validation of a novel and robust cell culture system in soybean (Glycine max (L.) Merr.) for promoter screening. Plant Cell Reports, 38, 1329-1345. doi: https://doi.org/10.1007/s00299-019-02455-5
Verdaguer B., de Kochko A., Beachy R. N. & Fauquet C. (1996) Isolation and expression in transgenic tobacco and rice plants, of the cassava vein mosaic virus (CVMV) promoter. Plant Mol Biol, 31 (6), 1129-39. doi: 10.1007/BF00040830.

1. A plant whose heritable genetic material comprises a gene encoding a transportable carbonic anhydrase protein under the control of a promoter active in root cells, such that the carbonic anhydrase is transported from the roots of the plant into the extracellular environment.

2. The plant according to claim 1, wherein the carbonic anhydrase is over-expressed in the extracellular environment of the root cells in comparison to a plant whose heritable genetic material does not comprise a gene encoding a transportable carbonic anhydrase protein under the control of a promoter active in root cells.

3. The plant according to claim 1, wherein the gene encoding the transportable carbonic anhydrase protein further comprises a signal peptide sequence for directing transport of the carbonic anhydrase into the extracellular environment of the plant root.

4. A polynucleotide sequence comprising a sequence coding for a polypeptide sequence under the control of a suitable promoter, wherein the polypeptide sequence comprises a carbonic anhydrase sequence and a signal peptide sequence, wherein the promoter is active in plant roots and capable of driving overexpression of the carbonic anhydrase, the signal peptide sequence being capable of directing transport of the carbonic anhydrase into the extracellular environment of a plant root.

5. The plant according to claim 3, wherein the signal peptide sequence is either natively encoded or translationally fused to the carbonic anhydrase sequence to ensure transport of the carbonic anhydrase to the extracellular environment of the plant root.

6. The plant according to claim 3, wherein the signal peptide sequence for directing transport is derived from a secreted protein that is transported to the extracellular environment by a protein secretion pathway.

7. The plant according to claim 3, wherein the signal peptide sequence is derived from the extensin protein.

8. The plant according to claim 1, wherein the promoter is derived from a gene from the same plant species or variety as the plant.

9. The plant according to claim 1, wherein the promoter is root-specific.

10. The plant according to claim 1, wherein the carbonic anhydrase further comprises a transmembrane domain or a membrane anchor sequence.

11. The plant according to claim 1, wherein the carbonic anhydrase is an α, β, γ, δ, ζ, η, θ, or ι subtype carbonic anhydrase.

12. The plant according to claim 1, wherein the gene encoding a transportable carbonic anhydrase protein comprises SEQ ID NO: 1 or SEQ ID NO: 2 or a sequence having at least 65% identity to SEQ ID NO: 1 or SEQ ID NO: 2, optionally wherein the promotor comprises SEQ ID NO: 6 or SEQ ID NO: 7 or a sequence having at least 65% identity to SEQ ID NO: 6 or SEQ ID NO: 7.

13. The plant according to claim 3, wherein the gene encoding a transportable carbonic anhydrase protein comprises SEQ ID NO: 1 or SEQ ID NO: 2 or a sequence having at least 65% identity to SEQ ID NO: 1 or SEQ ID NO: 2, optionally wherein the promotor comprises SEQ ID NO: 6 or SEQ ID NO: 7 or a sequence having at least 65% identity to SEQ ID NO: 6 or SEQ ID NO: 7; and wherein the signal peptide sequence comprises SEQ ID NO: 3 or SEQ ID NO: 4 or a sequence having at least 65% identity to SEQ ID NO: 3 or SEQ ID NO: 4.

14. (canceled)

15. A plant cell, cell line or progeny thereof comprising the polynucleotide sequence of claim 4.

16. The plant according to claim 1, wherein the plant is a crop plant selected from the group consisting of: corn, soybean, pea, cotton, canola, camelina, potato, tomato, sugar beet, cassava, sweet potato, alfalfa, wheat, barley, sorghum, oat, sorghum, millet, rye, teff, rice plant, or clover, cress, brassicas, vetch and prairie grasses, or a tree selected from the group consisting of poplar, spruce, pine, eucalyptus, oil palm and rubber.

17. (canceled)

18. The plant according to claim 1, wherein more carbon is sequestered into the extracellular environment of the plant in comparison to a plant whose heritable genetic material does not comprise a gene encoding a transportable carbonic anhydrase protein under the control of a promoter active in root cells.

19. A plant part, plant tissue, plant organ, plant cell, plant protoplast, embryo, callus, cell culture, pollen grain or seed, derived or obtained from the plant cell, cell line or progeny according to claim 15.

20. (canceled)

21. (canceled)

22. A bacterium comprising the polynucleotide of claim 4.

23. A plant comprising the polynucleotide of claim 4 stably integrated into the genome thereof.

24. (canceled)

25. A method for increasing the capacity of a plant to sequester carbon in the soil, the method comprising altering the heritable genetic material of the plant such that a carbonic anhydrase protein is transported from the roots of the plant into the extracellular environment.

26. (canceled)

27. (canceled)

28. A processed plant product obtained from the plant of claim 1, wherein the processed product comprises a detectable nucleic acid sequence of the gene.
